(19) United States
(12) Patent Application Publication
(10) Pub. No.: US 2004/0267340 A1
(43) Pub. Date: Dec. 30, 2004

(54) Modular thermal treatment systems with single-use disposable catheter assemblies and related methods

(75) Inventors: Iulian Cioanta, Weston, FL (US); Peter Gorky, Ft. Bragg, NC (US); Richard Barry Klein, Cary, NC (US)

Correspondence Address: BRADLEY M GANZ, PC, P O BOX 10105, PORTLAND, OR, 97296

(73) Assignee: WIT IP Corporation

(21) Appl. No.: 10/873,447

(22) Filed: Jun. 21, 2004

(30) Foreign Application Priority Data
Dec. 12, 2002 (WO) PCT/US02/39758

(51) Int. Cl. A61F 7/00; A61F 7/12

(52) U.S. Cl. 607/105; 607/113

(57) ABSTRACT

Methods, systems and computer program products include modular single-use disposable catheter assemblies with cassettes configured to matably engage with a console to circulate liquid to a treatment balloon in a closed loop circulation system. The console is portable, compact, and can be programmed to administer a plurality of different therapy types and to engage with different catheter configurations via the cassette body.

### RELATED APPLICATIONS

This application claims priority from U.S. Provisional Application Ser. No. 60/342,566, filed Dec. 20, 2001, and PCT Application Serial No. PCT/US02/39758, filed on Dec. 12, 2002, the contents of which are hereby incorporated by reference as if recited in full herein for all purposes.

### FIELD OF THE INVENTION

The present invention relates to systems and methods of delivering minimally invasive thermal therapies in a lumen or body cavity era subject and is particularly suitable for treatment of certain conditions of the prostate.

### BACKGROUND OF THE INVENTION

Conventionally, several types of thermal treatment systems have been proposed to treat certain pathologic conditions of the body by heating or thermally ablating targeted tissue. These thermal treatment systems have used various heating sources to generate the heat necessary to treat or ablate the targeted tissue. For example, laser, microwave, and radio-frequency (RF) energy sources have been proposed to produce the heat which is then directed to the targeted tissue in or around the selected body cavity. Thermal treatment systems have been, used to thermally ablate prostate tissue as well as to thermally treat or ablate the tissue of other organs, body cavities, and/or natural lumens.

U.S. Pat. No. 6,216,703 describes certain thermal treatment systems (including microwave energy systems) that can allegedly be used to treat both prostatitis and BPH (benign prostatic hyperplasia). The contents of this patent are hereby incorporated by reference as if recited in full herein. However, BPH and prostatitis, while both disorders of the prostate, are themselves distinct and different conditions and each typically is treated with different treatment strategies and therapies. Additional discussion of prostatitis and suitable treatments is found in co-pending and co-assigned U.S. Provisional Patent Application Ser. No. 60/308,344, entitled, Methods of Treating Prostatitis, the contents of which are hereby incorporated by reference as if recited in full herein.

One particularly successful thermal ablation system known as the Thermoflex® System (available from ArgoMed, Inc., of Cary, N.C.) used to treat BPH ablates the prostate by a thermocoagulation process. This thermal ablation system employs a closed loop liquid or water-induced thermotherapy (WIT) system which heats liquid, typically water, external to the body and then directs the circulating heated water into a treatment catheter. The treatment catheter is inserted through the penile meatus and held in position in the subject prior to initiation of the treatment to expose localized tissue in the prostate to ablation temperatures. The treatment catheter includes an upper end portion which, in operation, is anchored against the bladder neck and an inflatable treatment segment which is held relative to the anchored upper end portion such that it resides along the desired treatment region of the prostate. In operation, the treatment-segment expands, in response to the captured circulating fluid traveling therethrough, to press against the targeted tissue in the prostate and to expose the tissue to increased temperatures associated with the circulating liquid, thereby thermally ablating the localized tissue at the treatment site.

As an acceptable alternative to surgery (transurethral resection of the prostate (TURP)), the use of WIT (water-induced thermotherapy) has been shown to be a successful and generally minimally invasive treatment of BPH (benign prostatic hyperplasia). Generally stated, the term “BPH” refers to a condition wherein the prostate gland enlarges and the prostatic tissue increases in density which can, unfortunately, tend to close off the urinary drainage path. This condition typically occurs in men as they age due to the physiological changes of the prostatic tissue (and bladder muscles) over time. To enlarge the opening in the prostatic urethra (without requiring surgical incision and removal of tissue), the circulating hot water is directed through the treatment catheter which is inserted into the penile meatus up through the penile urethra and into the prostate as described above. The treatment segment expands with the hot water held therein to press the inflated treatment segment against the prostate, which then conductively heats and thermally ablates the prostatic tissue. For BPH therapies, the circulating water is typically heated to a temperature of about 60′-62° C. and the targeted tissue is thermally treated for a period of about 35-45 minutes to locally kill the tissue proximate to the urinary drainage passage in the prostate and thereby enlarging the prostatic urinary passage.

The closed loop WIT system and other circulating liquid thermal therapy systems employ components formed of flexible materials such as relatively thin flexible catheters with elastomeric treatment balloons and tubing that can relax over the course of the treatment due to their exposure to conditions associated with the delivery of the therapy (including system pressures and/or heat) when the therapy is administered over relatively long treatment times. Additionally, there can be a physiologic response to the treatment, and the size, resiliency, and/or density of the tissue in the treated region surrounding the prostatic urethra may also alter during the treatment (albeit somewhat differently in different subjects based on individual variation in tissue properties). For example, during ablation treatments, the necrosis of the localized treated tissue about the treatment balloon is such that the tissue in this region effectively shrinks. In the past, to attempt to compensate for this phenomenon, additional amounts of liquid were added in bulk to the closed loop circulating system at one point during the thermal therapy to attempt to boost lost pressure.

There remains a need to provide improved thermal therapy systems, particularly improved circulating fluid thermal treatment systems that are compact, economic, easy to use, and configured for improved field maintenance capability.

### SUMMARY OF THE INVENTION

Embodiments of the present invention are directed to providing modular treatment systems, computer program products, methods and devices with enhanced operational features. The modular systems include control consoles that are compact and relatively lightweight and catheter assemblies that are configured with a modular cassette that is configured so as to releaseably matably connect to the console during use.

The system can be configured to provide economic, lightweight, portable circulating liquid closed loop thermal therapy systems with automated pressure monitoring and fine adjustment capability to be able to selectably control the pressure and temperature of the liquid in the closed loop system over the course of the treatment. The console can be configured as a vertical-rise housing with a reduced footprint to reduce the amount of square feet of real estate used in clinician or hospital facilities. In certain embodiments, the modular cassette is a substantially rigid body that is attached to the catheter and configured with a catheter repeat-use deterrent system (to promote hygienic sterile single uses).

The systems, methods, and devices can be configured with other enhanced operational features, including one or more of: (a) a plurality of pre-programmed selectable treatment procedures for different physiologic treatment conditions, each of the procedures can have different therapy times, temperatures, and pressures (in certain embodiments, each selectable procedure can have up to about five different steps, and the system can be pre-programmed with four different procedures); (b) automatic inflation and/or deflation of the treatment balloon before insertion and/or removal (at the end of the treatment) to inhibit insertion and/or removal of the catheter into or from the subject when the treatment balloon is in an inflated state; (c) automatic smart chip or other repeat-use deterrent means, or automatic switch open/short at end of a procedure of the catheter assembly, to prevent or inhibit the re-use after the catheter assembly has been exposed to certain thermal thresholds; (d) infrared temperature sensors for the circulating fluid, (e) temperature safety override (optic coupling sensor for the pump motor and heating element sensors) as well as circulating fluid temperature sensor(s); (f) front panel mounted prominent emergency shutoff switch; (g) light weight console (less than about 8.5 kilograms); (h) self-diagnosing trouble shooting program cycle; (i) accepts user input to accept patient identifier and automatically generates a patient record of treatment (electronic and/or paper form); (j) graphic display of status of treatment on monitor with two dynamic graphs of time and temperature; (k) downloadable treatment summary capability (local or remote monitoring of number and type of procedures rim); (l) high speed pump-speed air purge cycle (200 rpm or twice the heating pump speed) to automatically prepare the system for circulating fluid without requiring hand pumping/manipulation of the treatment balloon; (m) disposable heating element in the cassette; (n) adjustable power input capacity having automatic recognition of 110 V 60 Hz or 220 50 Hz AC cycle converted to 24VDC to power the system; (o) configured with separate chambers to facilitate electromagnetic shielding of graphic components from pump motor or power source; (p) orienting board component connectors on the same side in the console to be accessible via the back panel for easy field service access; (q) audible alerts at desired points in the treatment and/or for a malfunction; (r) easy external access port for software upgrades and/downloads; (s) environmental condition monitoring sensors (temperature humidity); (t) back-up battery power supply to reduce operational disruptions; (u) surge protector to help maintain a level power supply input; and (v) pressure monitoring and control of the system during treatment.

Certain embodiments of the present invention are directed to closed loop fluid flow thermal treatment systems. The system can include a control console and a treatment catheter assembly. The treatment catheter assembly includes a catheter, a length of flexible conduit, and a cassette housing. The flexible conduit extends between the catheter and the cassette housing and, with the catheter, defines the circulating flow path of the liquid used to apply the thermal treatment in the catheter. The cassette housing is sized and shaped to be releaseably attachable to the console.

The treatment catheter can include a circulating liquid inlet channel, a circulating liquid outlet channel, and an expandable treatment balloon in fluid communication with the circulating inlet and outlet channels. In certain embodiments, the treatment catheter includes a region of increased insulation relative to the other portions.

The console includes a controller, a pump, user input keypad, operational circuitry, display, and power source. The console includes a cassette-receiving surface that is configured to matably receive and hold the cassette so that the pump is able to operably engage therewith.

The cassette housing is configured to securely hold a length of conduit that forms a portion of the closed loop fluid circulation flow path as well as selected electronic circuitry. The conduit is held in the cassette housing such that it is encased about a portion of the cassette housing, exits at a first location in the cassette, and then enters the cassette at a second spatially separate location so as to provide an externally accessible length of conduit. The externally accessible length may have a curvilinear configuration.

The pump has a pump head that is mounted on an external face of the console and mates with the externally accessible length of the conduit that extends from the cassette housing to thereby circulate the fluid in the liquid flow path of the circulating system. The pump can be operably associated with one or more stepper motors that can adjust the pressure in the closed loop system during operation (such as by adding or removing liquid and/or by adjusting the pump rotation speed (rpm)).

The controller has computer program code for (a) activating the pump, the heater, the temperature sensors, the pressure sensor and the pressure adjustment device to substantially continuously circulate heated liquid through the liquid circulation path; and (b) automatically adjusting the pressure in the liquid circulation path to compensate for operational pressure losses or to operate the system at a selected pressure at a desired time. The adjustment can be carried out to account for any physiological changes in the tissue proximate the targeted treatment region (such as in the prostatic urethra) so that the system maintains at least one selected operating pressure during administration of the thermal therapy. In certain embodiments, the system is configured to accept user input in situ to set the desired operating pressure(s), and other embodiments a series of increasing pressures are used to apply an increased pressure concurrently with heat at the target site in the body.

The controller can also have computer program code for (a) disabling operating circuitry in the cassette after the treatment catheter has been used for one treatment procedure (making the cassette and catheter a disposable assembly to promote single-use catheters); (b) automatically deflating the treatment balloon (before introduction or removal of the catheter); (c) activating a high speed air bubble purge procedure to filter air bubbles from the circulation path; (d) generating a patient record of treatment; (e) providing predetermined selectable treatment protocols; (f) performing a trouble shooting self-diagnostic test; and (g) automatically recording treatment parameters associated with the delivery of a patient's therapy.

The systems, methods, and computer program products can be used to treat urinary or prostate disorders or conditions such as prostatitis, BPH, or cancer, or to treat other tissues adjacent or proximate a natural body lumen or cavity. In certain embodiments, the pre-programmed treatment protocols can be for different therapies associated with different conditions of the prostate including both BPH and prostate. In certain particular BPH treatment embodiments, the circulating liquid can be heated to 57°-62° C. or higher external of the subject and directed into the treatment catheter at an inlet temperature of above about 57°-62° C. or higher for at least about 10-20 minutes.

Certain embodiments of the present invention are directed toward ambulatory thermal treatment systems having a closed loop liquid circulation path. The systems can include: (a) a portable control console having opposing front and rear portions and a cassette mounting region; and (b) a cassette housing sized and configured to be releaseably mounted to the console at the console mounting region. The console includes a power supply, a controller operably associated with the power supply, and a pump operably associated with the controller. In operation, the pump is configured to circulate liquid through a closed loop circulation path to administer a desired thermal therapy to a patient. The cassette is sized and configured to be releaseably mounted to the console at the console mounting region and the cassette houses a portion of the closed loop circulation path.

The cassette can include a length of flexible conduit held therein which forms a portion of the closed loop circulation path. During use, a portion of the conduit is held in the cassette so as to be in communication with the pump when the cassette is mounted to the console.

In particular embodiments, the length of conduit in the cassette is defined by portion of the length of flexible tubing that extends beyond the perimeter of the cassette housing.

Other embodiments are directed to closed loop circulating liquid thermal treatment apparatus. The device includes: (a) a portable console having a mounting surface with a power interface region thereon, the console comprising a controller, pump, and power supply therein, the power supply being in electrical communication with the power interface region on the console; and (b) a cassette housing having opposing front and rear primary surfaces, the rear surface including a power interface region, wherein the cassette housing is sized and configured to releasably mount to the mounting surface of the console so that the cassette power interface region engages with the console power interface region to be in communication with the controller and power supply in the console. The cassette holds a heating element therein. In use, the operation of the heating element is controlled by the controller in the console.

Still other embodiments are directed to single-use disposable cassettes for a thermal treatment system. The cassettes include: (a) a cassette housing configured with opposing front and rear surfaces, wherein the cassette is sized and configured to be releasably mounted to a control console that controls the administration of a thermal therapy to a subject; (b) a pressure sensor held in the cassette; (c) a cylindrical heater having a central liquid flow channel therethrough held in the cassette; (d) a temperature sensor operably associated with the heater held in the cassette; {e) an externally accessible power interface connection operably associated with the heater, temperature sensor, and pressure sensor, and (f) a length of flexible conduit held in the cassette defining a portion of a circulating liquid flow path, a portion of the length of flexible conduit being in communication with the heater.

Additional embodiments are directed to single-use disposable catheter assemblies. The catheter assemblies include: (a) a modular cassette housing having opposing front and rear surfaces, the cassette housing a portion of a circulating liquid flow path; and (b) a treatment catheter configured for insertion into the natural lumen or body cavity of a subject, the treatment catheter having an expandable treatment balloon thereon. The treatment catheter defines another portion of the circulating liquid flow path and is in fluid communication with the portion of the liquid circulating flow path held in the cassette. The front surface of the cassette includes: (a) an air bubble filtration button that selectably controllably alters the configuration of the circulation flow path of liquid flowing through the cassette; (b) a pressure sensor held in the cassette so that it is in communication with the circulating liquid flow path; (c) a pressure adjustment syringe having an associated plunger held in the cassette so that syringe is in fluid communication with the circulating liquid flow path; (d) at least one infrared sensor window formed in the rear surface of the cassette housing; (e) a pressure adjustment slot formed in the rear surface of the cassette housing proximate the pressure adjustment syringe. In operational position, the slot is adapted to receive a translating member therein that advances and retracts the plunger of the syringe.

Certain embodiments of the present invention are directed toward methods of treating a subject (such as, for example, the prostate of a subject) using a closed loop thermal treatment system having a console configured to receive and secure a modular cassette member thereon The method includes: (a) providing a portable console of a thermal treatment system having a power source, a pump, and electronic circuitry therein; (b) mounting a modular cassette member having a length of flexible conduit onto the console so that a portion of the length of flexible conduit engages with the pump in the console; (c) securing the cassette to the console; (d) accepting user input to select at least one of a plurality of different types of pre-programmed thermal therapy procedures; and (e) removing the cassette from the console after termination of the selected thermal therapy procedure.

### BRIEF DESCRIPTION OF THE DRAWINGS

The accompanying drawings, which are incorporated in and constitute a part of the specification, illustrate embodiments of the invention and, together with the description, serve to explain principles of the invention.

FIG. 1 is a front perspective view of a closed loop circulating treatment system with a console and catheter assembly according to embodiments of the present invention.

FIG. 2 is a side perspective view of the console and catheter assembly shown in FIG. 1, with the catheter assembly mounted onto the console according to embodiments of the present invention.

FIG. 3 is an enlarged view of the console shown in FIG. 1.

FIG. 4 is a side perspective view of the console shown in FIG. 1.

FIG. 5 is a rear view of the console shown in FIG. 1.

FIG. 6A-6C illustrate the device of FIG. 1 in a series of operative configurations. FIG. 6A illustrates the pre-assembly configuration. FIG. 6B illustrates the cassette being positioned on the console and FIG. 6C illustrates liquid being added to the circulation flow path according to embodiments of the present invention.

FIG. 7A is an enlarged partial side view of the cassette being aligned with the console for attachment thereto according to embodiments of the present invention.

FIG. 7B shows the device of FIG. 7A with the cassette being held in its mounted position against the console according to the embodiments of the present invention.

FIG. 7C shows the three operative positions of a control knob.

FIG. 8 is a side view of the device shown in FIG. 1 with the housing cutaway.

FIG. 9 is an opposing side view of the device shown in FIG. 8.

FIG. 10 is an enlarged front perspective view of the device shown in FIG. 8 and with the cassette cutaway.

FIG. 11 is an enlarged view of the device shown in FIG. 8 with the cassette cutaway.

FIG. 12 is a rear view of the device shown in FIG. 8.

FIG. 13 is a front perspective view of a stepper motor subassembly (to control the system pressure) shown in position in FIG. 12.

FIG. 14 is a rear view of a portion of a console housing illustrating two separate vertically stacked enclosures according to embodiments of the present invention.

FIG. 15 is an enlarged front view of the cassette shown in FIG. 1.

FIG. 16 is an enlarged rear view of the cassette shown in FIG. 15 according to the embodiments of the present invention.

FIG. 17 is a top view of the cassette shown in FIG. 15 with the front cover removed.

FIG. 18 is a top view of the other side of the cassette shown in FIG. 17 with the back cover removed.

FIG. 19 is a front three-dimensional perspective view of the device shown in FIG. 17.

FIGS. 20A and 20B are schematic illustrations of left and right sides of operating circuitry of a console and plug-in disposable cassette assembly according to embodiments of the present invention.

FIG. 21 is pressure control circuit diagram for controlling the pressure in a closed loop system according to the embodiments of the present invention.

FIG. 22 is a power interface circuit diagram according to the embodiments of the present invention.

FIG. 23 is a circuit diagram of a controller interface for auxiliary wire panel according to embodiments of the present invention.

FIG. 24 is a heater/pressure sensing circuit diagram according to embodiments of the present invention.

FIG. 25 is a pump control circuit diagram according to embodiments of the present invention.

FIG. 26A is a circuit board diagram of a heater/pressure sensor board according to embodiments of the present invention.

FIG. 26B is another view of the board shown in FIG. 26A illustrating the power connection interface according to embodiments of the present invention.

FIG. 27A is a front view of a portion of the pump (open to receive the conduit therein) and a portion of the circulation flow path and associated operating components according to embodiments of the present invention.

FIG. 27B illustrates the device shown in FIG. 27A with the pump in operative position.

### DETAILED DESCRIPTION OF EMBODIMENTS OF THE INVENTION

The present invention now will be described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fly convey the scope of the invention to those skilled in the art. In the figures, certain elements, regions, or features may be exaggerated for clarity. Like numbers refer to like elements throughout. Also in the figures, broken lines, where used, indicate optional features, operations, or components.

The thermal treatment systems of the present invention may be configured to administer thermal therapies of any desired temperature (cooled and/or heated) in the cavity or natural lumen in the subject's body. For cooling, the thermal treatment systems may be configured to expose the targeted tissue to temperatures below the average body temperature, such as to below about 15°-20° C. (including about 0° C.). For heating, the thermal treatment systems can be configured to expose the targeted tissue to temperatures heated to non-ablation temperatures (below about 45° C.) or above ablation temperatures (such as above 45° C.). The system can be configured to operate at selectable temperatures. In certain embodiments, the system can be configured with a relatively wide selectable range of temperatures. For example, the system can be configured to provide circulating liquid that can be selectively heated to one or more operating temperatures in a range of between about 50°-80° C. during a single treatment. The present invention finds use for both veterinary and medical applications. The present invention may be advantageously employed for treatment of subjects. “Subjects,” according to the present invention, include animal subjects, and are preferably mammalian subjects (e.g., humans, canines, felines, bovines, caprines, ovines, equines, rodents, porcines, and/or lagomorphs), and are preferably human subjects.

In certain embodiments, the thermal treatment system is a thermal ablation treatment system configured to substantially continuously circulate fluid heated to above about 45° C. (and typically to about 57°-62° C.) for at least a portion of the thermal therapy. Thus, the term “thermal ablation” refers to exposing the targeted tissue to a temperature that is sufficient to kill the tissue. The thermal ablation can be carried out by causing thermocoagulation in targeted tissue via contact with an expandable treatment balloon on a catheter inserted into the subject which is configured to direct circulating hot liquid heated external of the body of the subject to the targeted treatment region within the biological subject.

For ease of discussion, the embodiments of the present invention will be primarily discussed for use in the male urethra. However, the catheters of the present invention may be alternately configured and adapted as appropriate for insertion in other natural lumens or body cavities such as, but not limited to, the colon, the uterus, the cervix, the throat, mouth or other respiratory passages, the ear, the nose, blood vessels, and the like.

In certain embodiments, the thermal treatment systems can be configured to administer thermal ablation therapy to treat BPH or thermal therapies to treat prostatitis. In treating BPH or prostatitis, the walls of the prostatic urethra can be thermally treated by contact with an expandable treatment balloon which expands responsive to the quantity of heated fluid circulating therein, as the fluid travels, captured in the treatment catheter.

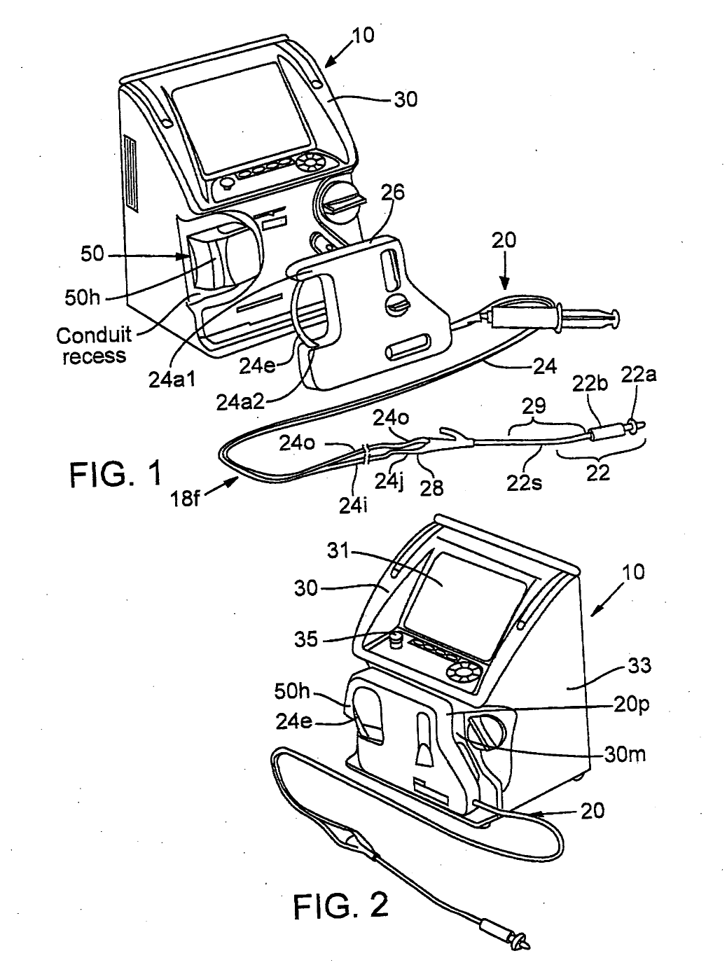

FIG. 1 illustrates a closed loop circulating fluid thermal treatment system 10 having a catheter assembly 20 and a control console 30. The control console 30 and the catheter assembly 20 are configured to be releasably matable, so as to engage during operation and disengage upon completion of the treatment. The catheter assembly 20 can be configured as a single-use disposable device. The catheter assembly 20 is configured to engage or mount to the console 30 without requiring intensive labor efforts. The electronic connections and interfaces between the console and catheter assembly 30, 20, respectively, automatically electrically engage when the cassette 26 is properly mounted to the console 30.

The control console 30 houses the primary operational circuitry, program code, power source, pump, and controller of the system 10. The catheter assembly 20 includes a catheter 22 (the distal portion of which is configured for insertion into the natural lumen or body cavity of a subject), a length of elastomeric or compressible conduit 24, and a cassette 26. As shown, the cassette 26 is configured to encase a portion of the length of the conduit 24 and to allow a portion of the conduit 24e to be externally accessible. The externally accessible portion of the conduit 24e is configured to engage with the pump head 50h of the pump 50 when the cassette 26 is mounted to the console 30. The pump 50 is a peristaltic pump with rollers that compress the liquid in the conduit to circulate the liquid in the closed loop system. As such, in proper position, the pump head 50h is able to firmly abut the conduit 24e to compress the conduit 24e in response to a desired pump speed (typically described in a rate of revolutions per minute (rpm)). A suitable pump identified by part number 313D (a three roller peristaltic pump with product code 033.3411.000) is available from Watson Marlow, Inc., of Paramus, N.J.

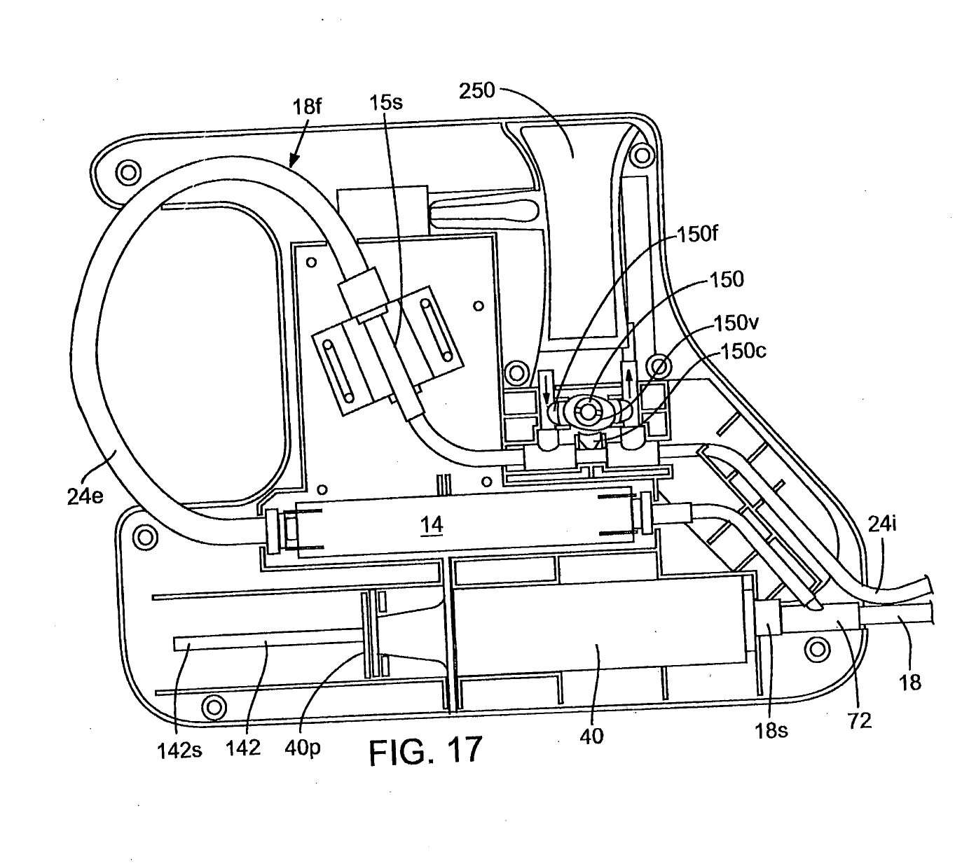

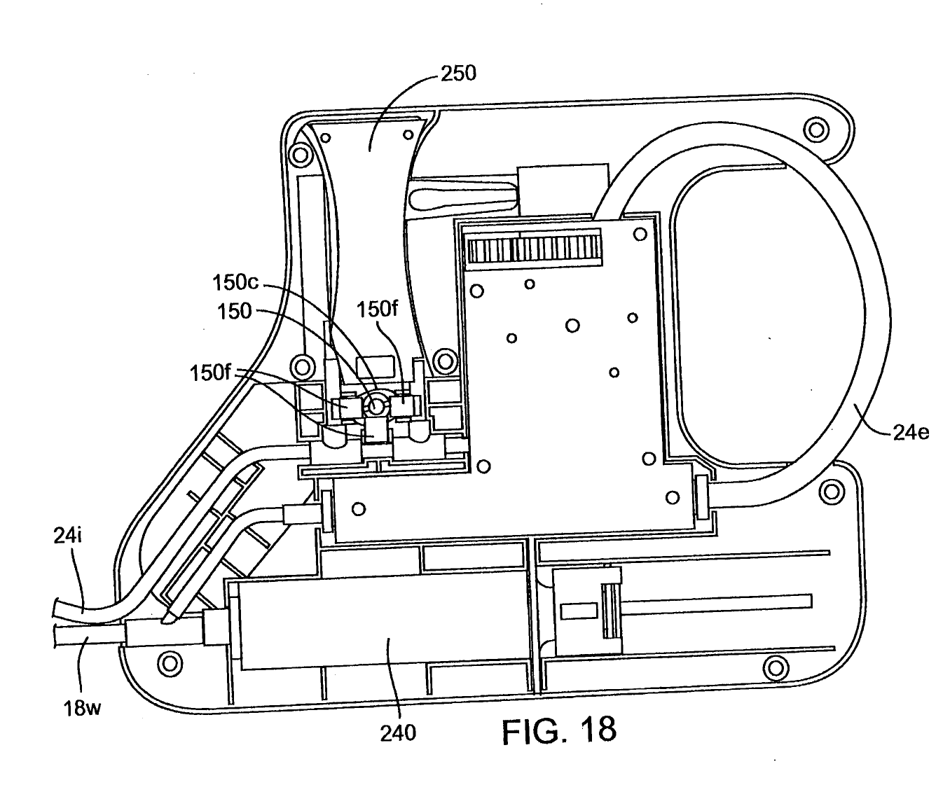

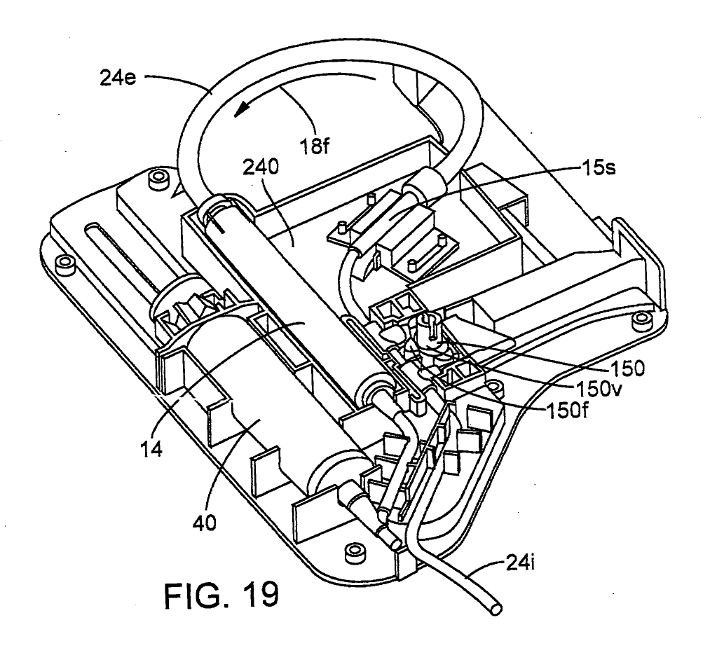

To help identify when the pump 50 and conduit 24 are not properly engaged, a contact sensor may be disposed proximate the connection that can generate an audible alert to indicate when there is not proper engagement and/or alignment. Such a contact sensor can help facilitate proper fluid flow and thus, inhibit overheating in the heater 14 (FIG. 17) in the cassette 26. An example of a suitable contact sensor is a photo IC output photo-microsensor or opto-coupler such as those available from OMRON of Schaumburg, Ill. identified by part number family series EE-SX4009-P1/P10.

During active thermal therapies, the pump 50 may be set to operate at about 100 rpm, while during cooling the pump may be set to operate at between about 130-150 rpm. During air bubble filtration, when desired (typically at start-up and before initiation of the procedure), the pump 50 may be set to operate at a high purge pump speed, such as greater than about 150-200 rpm. The purge pump (bubble reducing) cycle may be set to run automatically upon power-up or at the start of a treatment cycle (pre- and/or post-catheter insertion into the body of the subject).

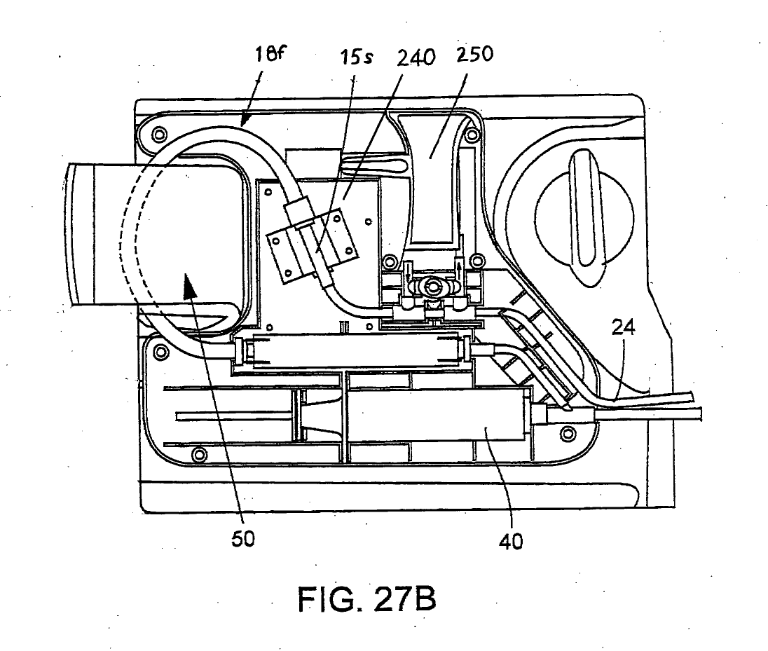

As shown, the tubing or conduit 24 extends from the catheter, a distance in the body of the cassette 26, exits at a first location on the cassette 26a₁, enters at a second location 26a2 spaced apart from the first location 26a1, and extends out of the cassette 26 and connects to the catheter 22. Thus, the catheter 22 and the tubing or conduit 24 defines the primary circulation flow path 18f having an inlet channel 24i and outlet channel 24o with respect to the catheter 22 that in operation circulates the thermally treated liquid from and to the expandable treatment balloon 22b. In certain embodiments, the externally accessible pump engaging portion of the conduit 24e is curvilinear or arcuate and bridges two opposing members or arms 26a1, 26a2 of the cassette 26. FIGS. 27A and 27B illustrate the interconnection and alignment of the conduit 24e with the pump 50. As shown in FIG. 27A, a portion of the pump 50a translates away from a stationary portion 50b. A length of the conduit 24e is positioned between these portions and then portion 50a is closed to rest against portion 50b compressing the conduit 24e therebetween (FIG. 27B),

Thus, in certain embodiments, in operation, liquid is substantially continuously circulated in a closed loop system defined by the circulation path 18f. A portion of the circulation path is defined by the catheter 22 with an expandable treatment balloon 22b. In position, tissue in a targeted region in the lumen or natural cavity of a subject is contacted with the expanded treatment balloon to conductively administer a thermal therapy. Typically, the thermal therapy has a duration of at least about 5 minutes. The pressure in the closed loop system can be continuously monitored. The pressure can be automatically adjusted during the administration of the thermal therapy to increase the penetration depth of the therapy and/or to maintain the system at selected operating pressures responsive to desired treatment system pressure and/or physiologic changes in the treated tissue and pressure losses in the system over the course of the thermal therapy treatment The system 10 may also be configured to monitor and adjust the temperature of the circulating liquid during the treatment (increasing and/or decreasing over the delivery of the therapy) to administer a concurrent combination of heat and pressure therapy to targeted tissue.

Optionally, for thermal and/or thermal ablation therapies, the operations can be carried out so as to provide a series of different pressures over the course of a treatment. The system can also be configured to administer several different therapy procedures each corresponding to a particular condition. In this manner, the system 10 can be used for administering a plurality of different thermal therapies.

In certain embodiments, the system 10 can be configured to provide a plurality of different pressure and temperature combinations over the course of the treatment session. For example, the system 10 can be configured to automatically generate up to about five different temperatures and pressures so that the desired temperature and pressure is activated at the desired time in the treatment cycle. Additional temperature and pressure combinations can be employed where desired.

In certain embodiments, the system 10 can be configured with a selection menu that allows a clinician to select up to four, five or more different therapy types. The therapy type can correspond to the particular condition being treated. For example, the system 10 can be configured to treat BPH, prostatitis, UTI (urinary tract infection), uterine conditions, and the like. As such, the system 10 may be configured with a catheter assembly 20 identification system that inhibits the selection of a therapeutic program for a condition that does not correlate with the identification of the catheter 22. For example, selection of a female uterine (endometrium) therapy having a 5-minute high temperature (65°-85° C.) therapy duration will be inhibited when the catheter assembly 20 is identified as associated with a catheter 22 configured for the prostate. The identification may be by serial number, part number, assigned code name, or other suitable computer recognition or correlatable identification means. The system may require that the identification be entered manually at activation or the start of the procedure, or may be configured to automatically read the identification data such as by optic means including a bar code and/or scanner, smart data chip mounted on the catheter assembly 20, and the like.

As such, in certain embodiments, the system 10 can be pre-programmed to have a first system pressure during an initial portion of the therapy and then a second substantially constant (or increasing) system pressure of about 0.5-3 atm during a secondary portion of a thermal ablation heating sequence, the thermal ablation lasting at least about 5-20 minutes. In particular embodiments, the pressure in the system can be at about 0.75-2 atm, and typically at least about 1.0-1.5 atm during at least a latter or secondary portion of the treatment.

In certain embodiments, the system 10 can be configured to accept user input to increase or adjust the pressure to the patient's zone of comfort. The user input can include a limit or override (either a pressure stop and/or a ramp rate limiter) to assure that the system is not exposed to undue operating pressures. The user input may be accepted during a 5-10 minute initial heating portion of the thermal therapy, and/or during an elevated temperature portion of the thermal therapy (typically administered after about 5-10 minutes).

In certain embodiments, the pressure adjustment can be carried out during the thermal therapy so-that the operation is controlled to between about 0.1-0.5 psi resolution to inhibit pressure variation from planned pressures during at least selected portions of the active administration of the thermal therapy treatment. Maintaining pressures in the system at desired or constant operating pressures by substantially monitoring the system pressure in a manner that can take into account a particular patient's physiology as well as operating conditions may improve consistency between treatments, patient to patient.

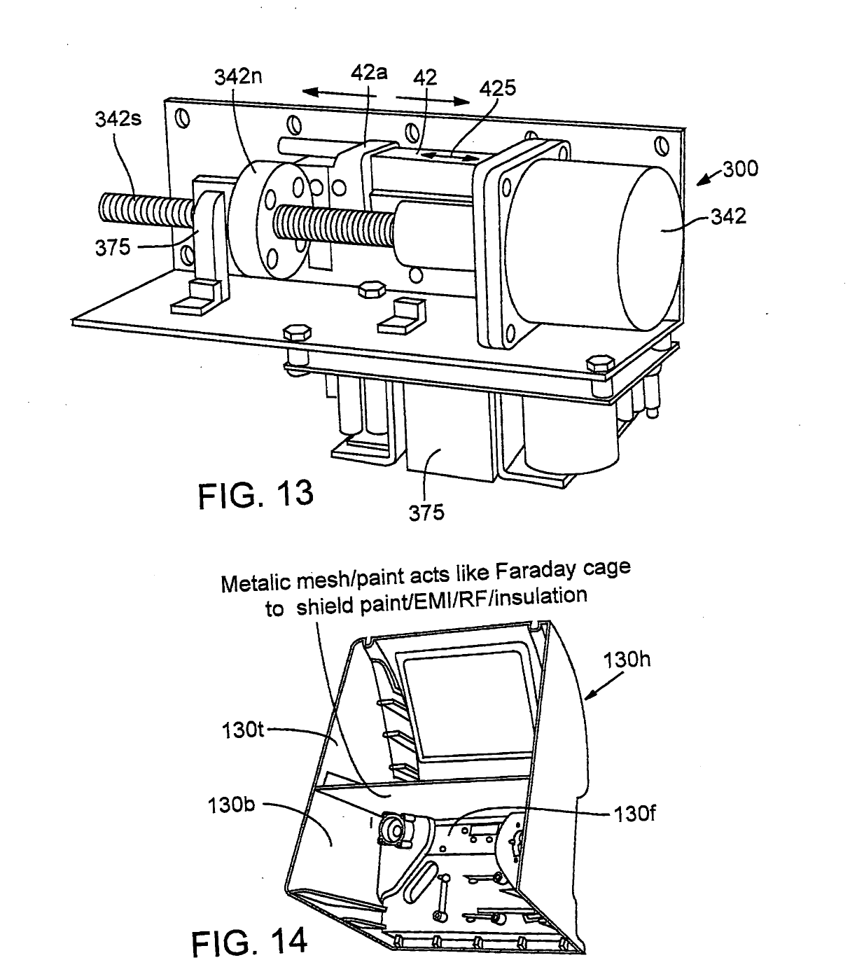

The pressure adjustment can be carried out by using a stepper motor (such as the one identified as 342 in FIG. 13) to automatically add or remove liquid from the volume circulating in the closed loop circulation based on the monitored pressure. In certain embodiments, the initial volume of circulating liquid can be on order of 100 ml or less, and liquid in the additional amount of 10-30% can be added over the at least 15 minutes of the thermal therapy treatment. In certain embodiments, an initial circulating volume of about 50 ml or less is circulated in the closed loop system; the typical amount of liquid added in over the course of the treatment can be on the order of about 5% or more.

In certain embodiments, a syringe 40 (FIG. 17) held inside the cassette 26 that is in fluid communication with the circulating flow pat 18f, can be used to both add and remove liquid from the system to help maintain the operational pressure substantial constant at a desired operating pressure(s). In certain embodiments, the system operating pressure (typically ranging from about 0.5 atm-7 atm or even larger) can be monitored and the pressure adjustment may be carried out to so as to maintain the pressure or adjust the pressure with a pressure resolution of between about 0.01-0.10 psi.

As is known to those of skill in the art, the treatment balloon/catheter 22b used to treat a particular subject can be custom-fit to have a length chosen to fit the length of the patient's prostatic urethra (typically chosen from a ranger of catheter sizes with treatment balloons ranging in length from about 1.5 cm to about 6 cm). The additional liquid added can be a multiple of the length of the treatment balloon, i.e., 1.5 ml, 3 ml, or 4.5 ml for a 1.5 cm treatment balloon and 6 ml, 12 ml or 18 ml for a 6 cm treatment balloon. As shown in the figures, the catheter 22 may also optionally include a bladder anchoring balloon 22a (FIG. 1). The catheter 22 may include regions with increased insulation and structural reinforcements as desired. See, e.g, U.S. patent application Ser. No. 10/011,700 filed Nov. 13, 2001, identified by Attorney Docket No. 9149-16, and related provisional application Ser. No. 60/248,109, the contents of which are hereby incorporated by referenced as if recited in full herein.

FIG. 2 illustrates the catheter assembly 20 mounted to the control console 30. In this embodiment, the rear primary source 20p of the cassette 20 mounts to a front surface mount portion 30m of the control console 30. The externally accessible conduit 24e is configured to wrap around and engage with the pump head 50h. FIG. 17 illustrates the internal components of the cassette 26 and one embodiment of a flow path 18f and will be described further below.

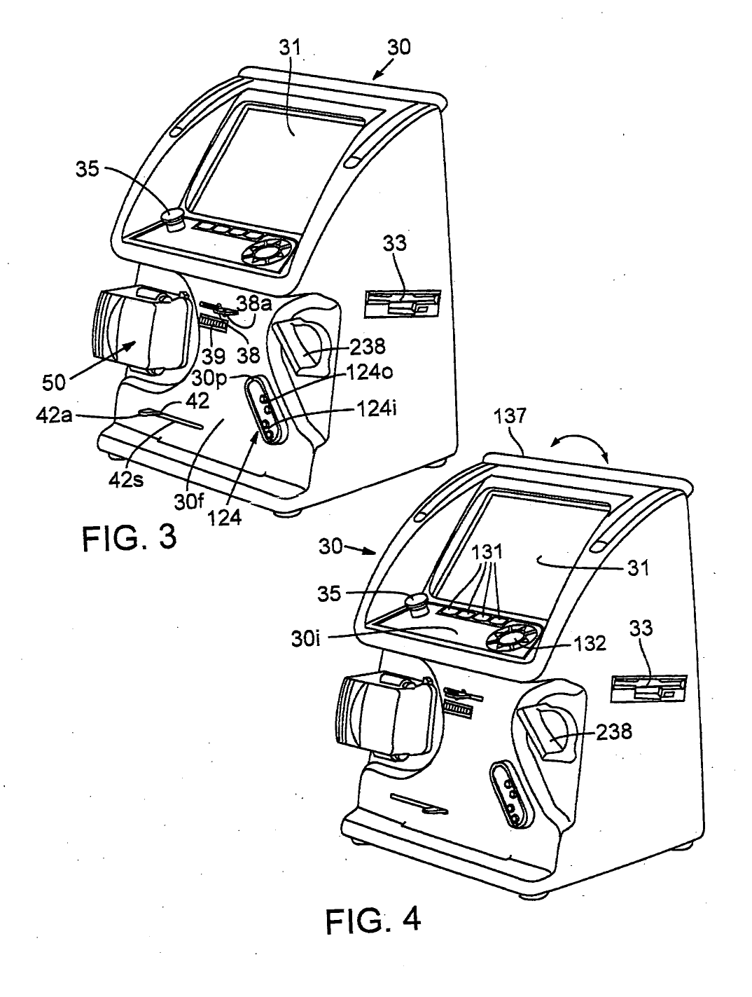

FIG. 3 illustrates the control console 30 without the cassette 26. As shown, the control console 30 includes a monitor or display 31, an externally accessible computer data port 33, and a prominent emergency shut off button 35. The front surface of the console 30f also includes three cooperating regions 38, 39, and 42, that operably engage with corresponding regions 138, 139, and 142 (FIG. 16) on the cassette 26, when the cassette is mounted onto the console 30.

The first cooperating region 38 can be configured with a securing and releasing arm 38a that extends a distance out from the console surface and is configured to attach to a locking mechanism 138m (FIG. 16) in the cassette 26. The arm 38a can be configured to move and engage with a locking or securing mechanism 138m of the cassette 26 (FIG. 16).

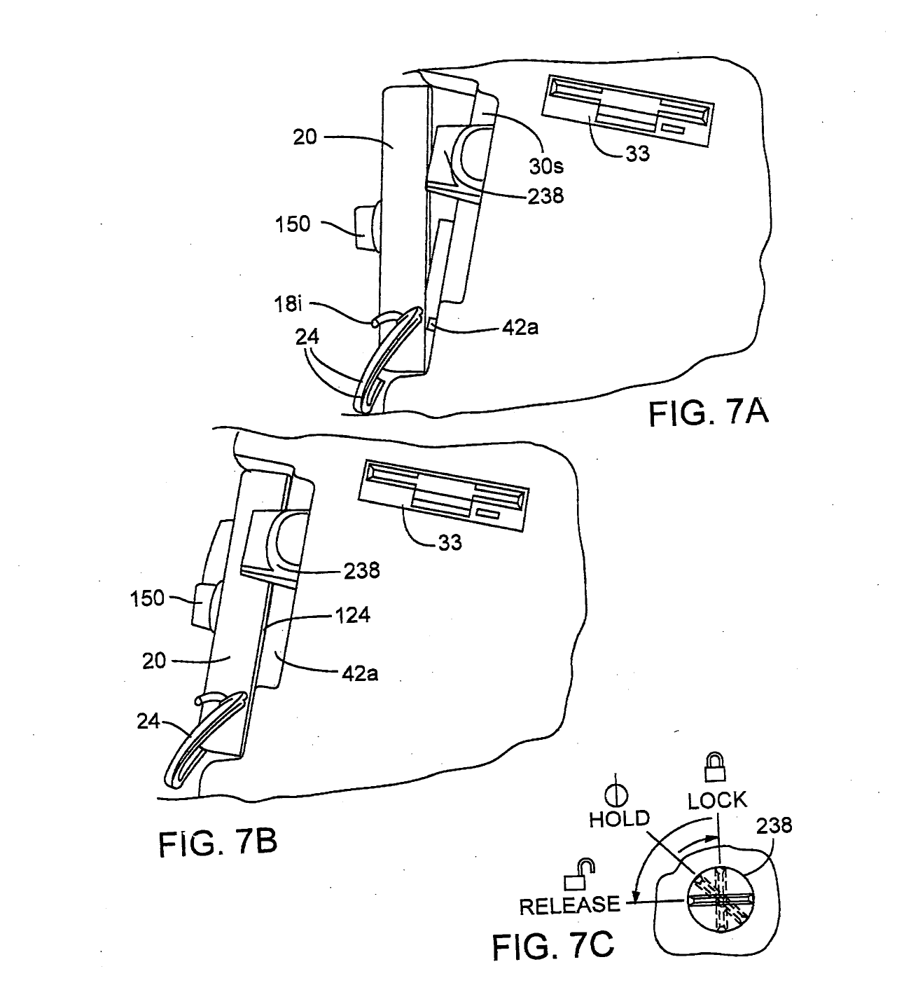

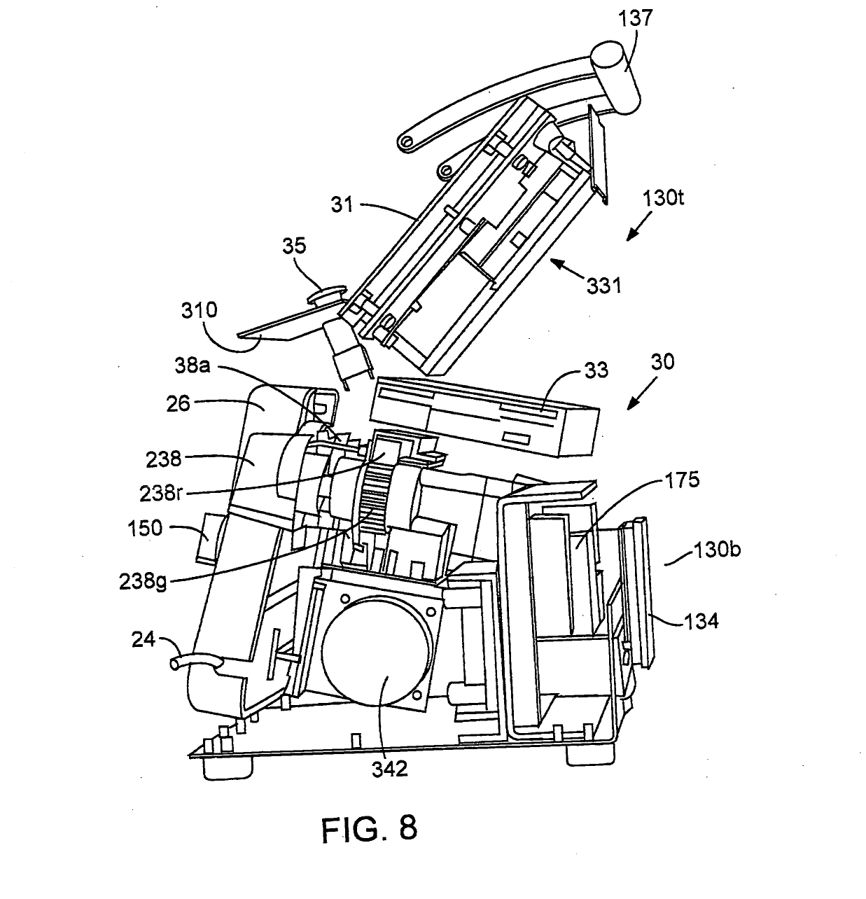

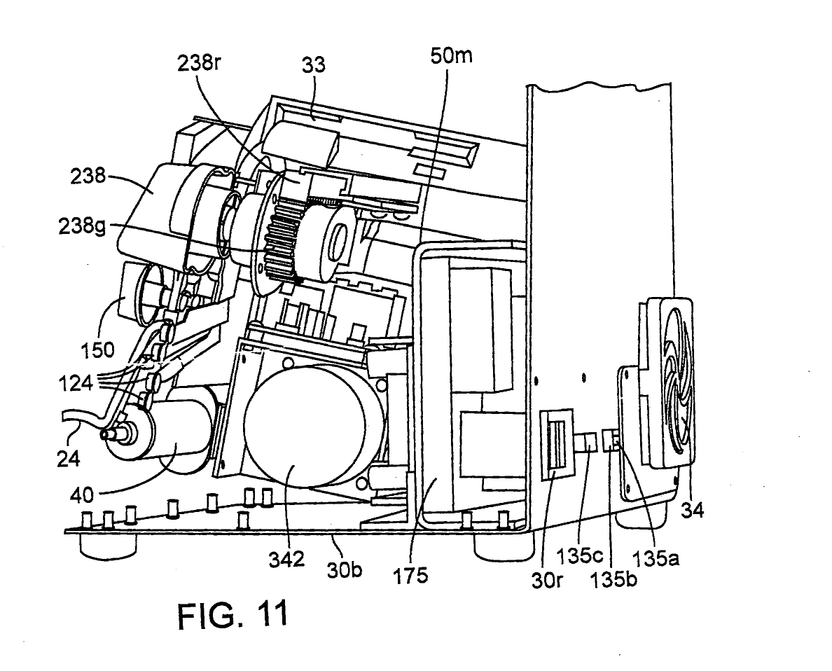

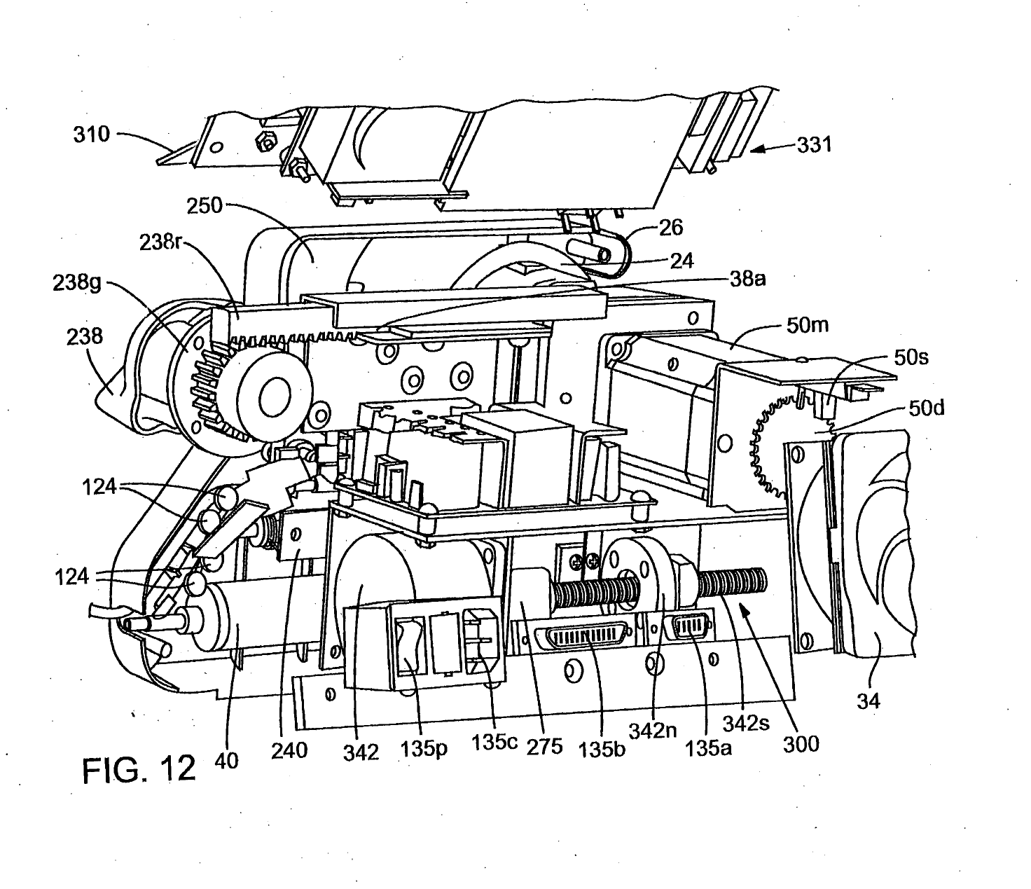

In certain embodiments, as shown in FIGS. 11 and 12, the arm 38a can be attached to a rack 238r that engages with and is controllably linearly driven by a pinion gear 238g that rotates responsive to rotation of the knob 238. As shown in FIG. 7C, the knob 238 can have three different angular operative positions, one corresponding to a null position where the rack and pinion gear system locates the locking mechanism 138m in a position that allows the cassette 26 to be positioned over the console 30, a second operative position that locks the cassette 26 to the cassette via arm 38a and locking mechanism 138a, and a third operative position where upon rotation to this position, the cassette 26 is released or forced away from the body of the console 30. FIG. 7A illustrates that during mounting the knob 238 can be positioned at 45 degrees. FIG. 7B illustrates that during use the knob 238 can be positioned at 90 degrees to lock the cassette against the console 30. As shown in FIG. 7C, the three operative positions of the knob 238 can be set at 45 degrees from horizontal for a null position, at 90 degrees for a lock position, and at 180 degrees for a release position. Other angular offsets may be used as desired as will be appreciated by those of skill in the art. The gear 238g (FIG. 11) rotates responsive to rotation of the knob 238. The gear 238g then linearly translates the rack 238r (FIG. 11), that, in turn, moves the cooperating locking arm 38a (FIG. 1) so that it engages or disengages with the locking mechanism 138m (FIG. 16) of the cassette 26 (FIG. 16). As such, in operation, the knob 238 is turned to the desired orientation (such as 90 degrees) to lock the cassette 26 is on the console 30 during use and then to release, the operator can rotate the knob 238 to a different orientation (such as 180 degrees) forcing the cassette 26 to move the arm forward and eject or release the cassette body 26 from the console 30 in response thereto.

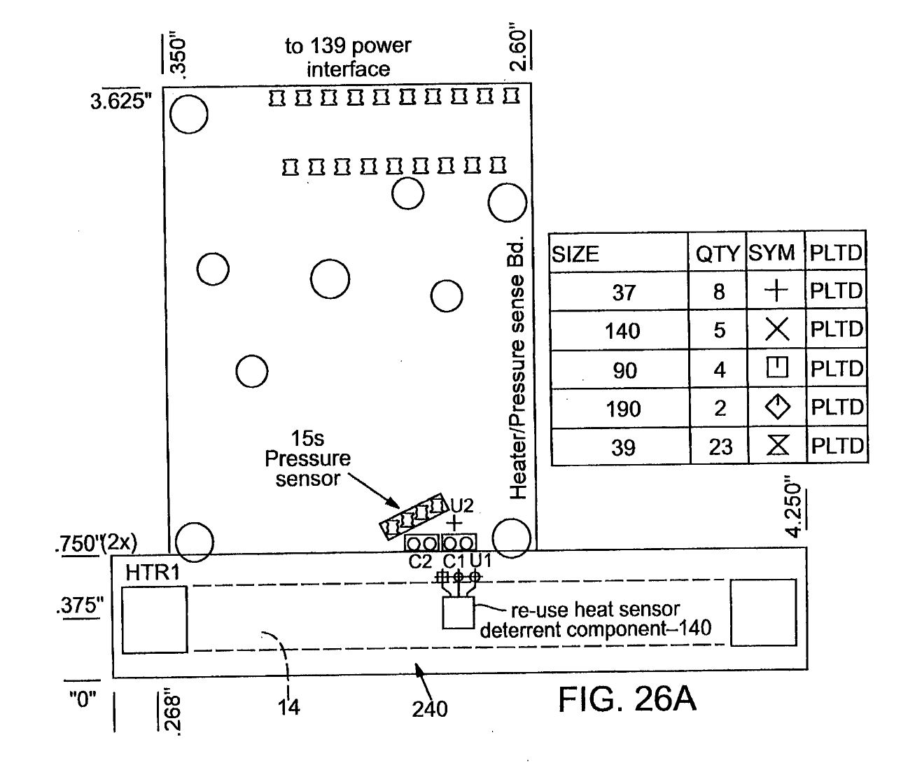

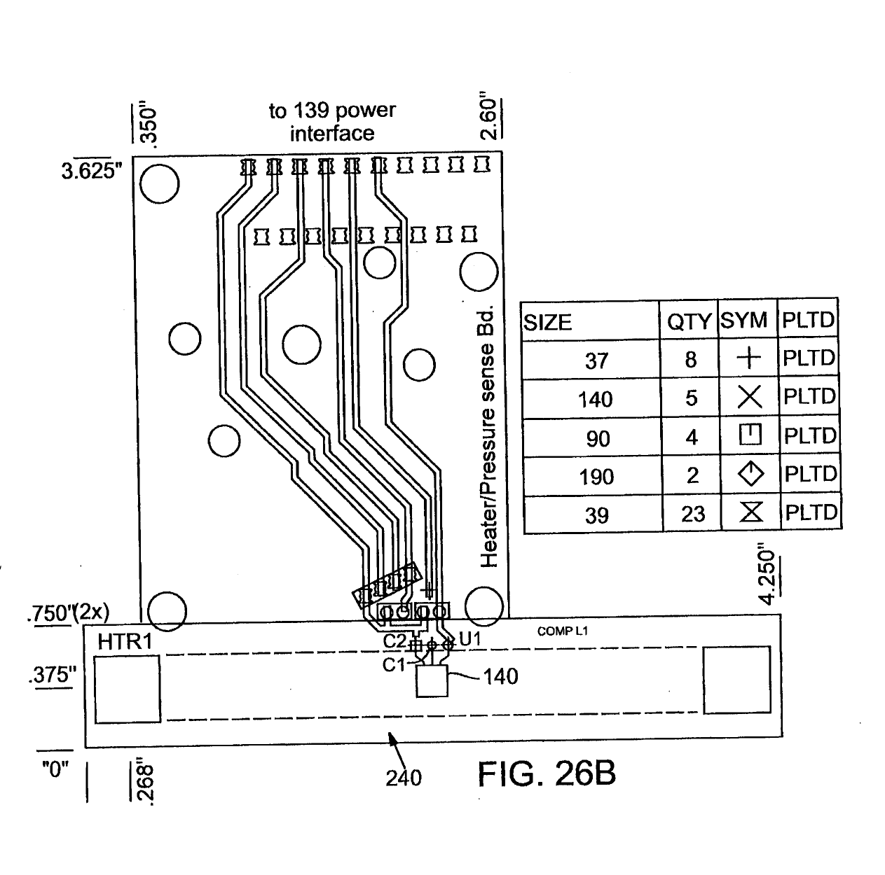

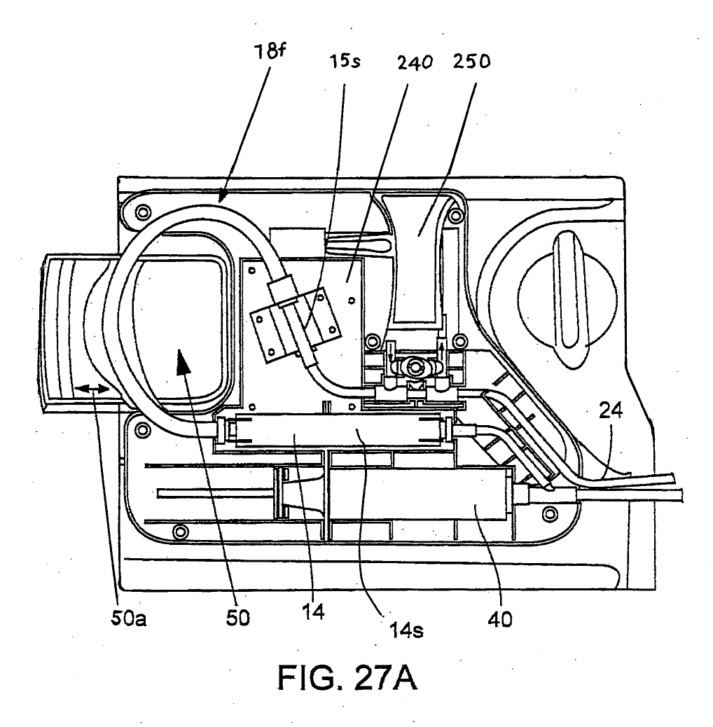

The second cooperating region 39 can be a power strip interface or connection that provides a power interface with the cassette 26. As shown in FIG. 16, the cassette 26 includes a power plug in connection 139p that is configured to operably engage with the power strip interface 39 on the console 30. FIGS. 26A and 26B illustrate that a circuit board 240 can be configured with traces that extend to connect to a power source in the console 30 at the power interface 139. The third cooperating region 42 is configured to engage with a pressure adjustment component comprising a syringe 40 positioned in the cassette 26. The third cooperating region 42 includes a slot 42s that aligns with a slot 142s on the cassette 26. An outwardly extending arm 42a on the console 30 extends out of the slot 42s on the console 42s and enters the slot on the cassette 142s to contact or engage with the plunger 40p of the syringe 40. The arm 42a is in communication with a stepper motor 342 (FIG. 13) that advances and retracts the arm 42a that in turn advances and retracts the syringe plunger 40p. In this manner, the plunger 40p can be substantially continuously adjusted to automatically and selectably advance and retract to add and remove liquid from the flow path 18f during operation.

FIG. 13 illustrates an exemplary embodiment a syringe or pressure adjustment assembly 300 that is housed in the console 30 so that the adjustment arm 42a extends outwardly from the slot 42s. The assembly 300 may also include position spatially separated location sensors 375 (one at each end of travel) to limit the travel distance of the plunger 40p to a desired length “L” (extending between the retracted and extended directions).

In certain embodiments, referring to FIG. 3, the front surface 30s of the console 30 may also include a plurality of infrared sensors 124, at least one sensor 124i configured to sense the temperature of the circulating liquid on the inlet path 24i through the conduit 24 and at least one sensor 124o configured to sense the temperature on the outlet path 24o through the conduit 24. The conduit 24 may be made of a translucent or transparent elastomeric material (at least the portion proximate the infrared sensor(s) 124). The infrared sensors 124 may be positioned on the console 30 on a protruding surface 30p. The protruding surface 30p can be configured so as to be matably receivable into a corresponding recess 130r (FIG. 16) on the rear surface 26r of the cassette when the cassette 26 is in position on the console 30. Still referring to FIG. 16, the recess 130r can be configured with one or more (shown as a plurality of windows) 130w that allows the infrared sensors 124 to be in optical communication when the cassette 26′ is attached to the console 30. The at least one window 130w may be defined by an appropriately sized aperture or slot located over the conduit 24 or may optionally be formed with a translucent or transparent material.

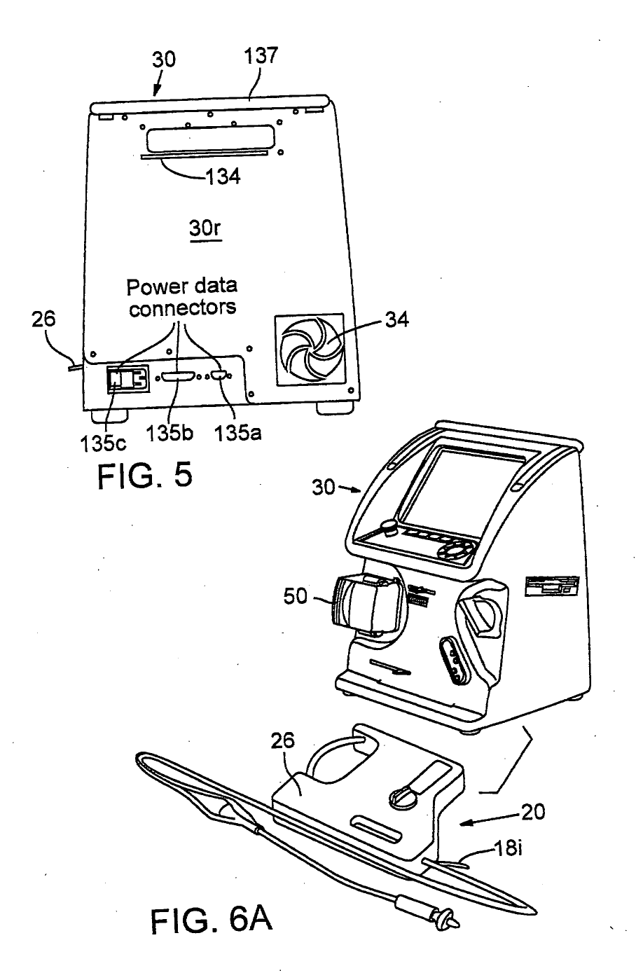

FIG. 4 illustrates that the console 30 may include a rotatable handle 137 that can be raised to carry the console 30 and then lowered to be substantially flush with the upper portion of the body contour of the console during use. FIG. 4 also illustrates that the console 30 can include a series of user input devices or keypads 30i. As shown, the input devices include a plurality of membrane switches 131 (that can operate as functional softkeys, the response or input of the keys varying based on the step in the procedure) and a membrane arrow scroll pad 132. These input devices 30i allow an operator to select pre-programmed treatment procedures or define in situ a desired treatment procedure protocol (temperature, times, and pressures, as desired) and also allows the clinician to enter patient data. The console 30 may also include a printer data port 135a (FIG. 5) to allow a clinician to download operation or patient record information.

FIG. 5 illustrates the rear side of the console 30. As shown, the console 30 includes a fan 34 located in fluid communication with the lower portion of the console 30 and an air vent 134 located in a top portion of the console 30. The rear wall 30r may also hold the AC power connection 135c and RS 232 connector 135b. As shown in FIG. 14, the console 30 can be configured with two separate compartments with a floor 130f extending between a top compartment 130t and a bottom compartment 130b. The bottom compartment 130b can be substantially electromagnetically (and/or vibrationally, heat, and RF) shielded from the top 130t. The top compartment 130t can hold sensitive or susceptible components (the graphic display, graphic display cards, circuit boards etc.) while the bottom compartment 130b can hold the fan 34, the power source, surge protector, and main mechanical mechanisms (syringe, locking mechanisms) the like. An air vent (not shown) can be formed into the floor 130f to allow air to circulate in the console. The air vent in the floor can be formed of a metallic mesh material and/or coated to inhibit RF or electromagnetic penetration. Similarly, the rear, side and front walls of the console as well as at least one primary surface of the floor 130f can be coated with a metallic coating to help shield the console (so as to form a Faraday cage).

FIGS. 8-12 illustrate different views of the interior components and their locations in the top or bottom compartment 130t, 130b, according to embodiments of the present invention. FIG. 8 illustrates one side view of the console 30 and cassette 26. As shown, the power supply 175 that converts the AC input power (that can accept either 110 or 220 AC) to 24DC is located in the rear of the unit. A medical grade power supply is available from CONDOR of California. The graphic display circuitry 331 is positioned in compartment 130t proximate the display 31. The user input overlay 310 is shown where the membrane switches are located.

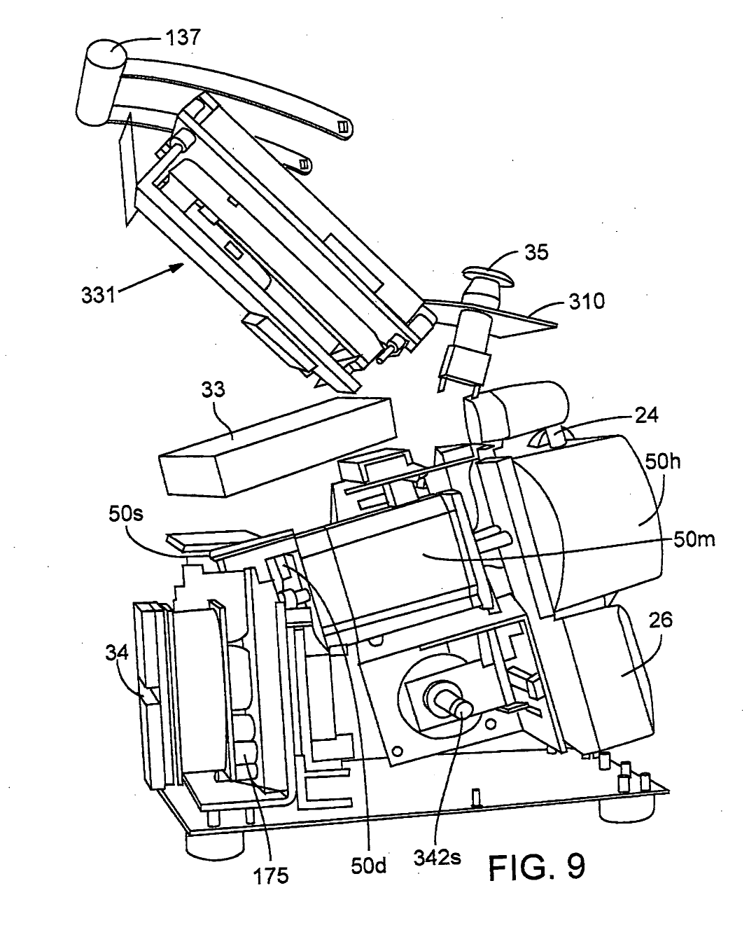

FIG. 9 illustrates the opposite side view of that shown in FIG. 8. The pump 50 includes a pump (stepper) motor 50m (24V DC) and is operably associated with a disc 50d with teeth located about its perimeter. A photo optical sensor 50s is positioned to extend over a perimeter portion of the disc 50d so as to be able to optically read and count the speed of rotation of the pump 50. The system 10 can be configured to generate an alert if the pump speed does not correspond with the selected mode of operation (such as if the pump has an irregular low speed indicating a potential malfunction that may lead to an overheating condition).

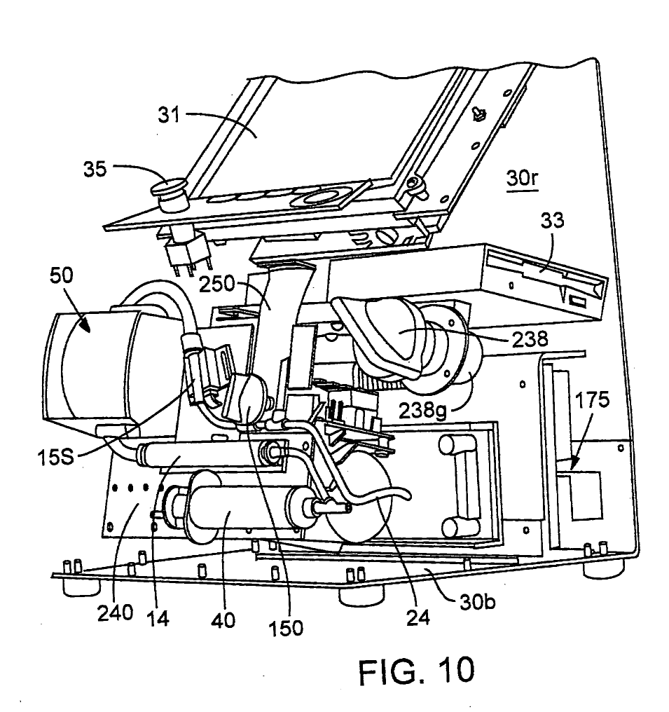

FIG. 10 is a front perspective view with the cassette housing (front and back covers or walls) removed. As shown, the console 30 includes a rear metal wall 30r and a metal floor 30b. FIG. 11 illustrates a partial side view enlarged relative to the view of FIG. 8. FIG. 12 is a partial view of the back of the console 30 with the back wall removed. An on off power switch 135p is shown as well as a DC-DC power supply 275 that steps down the voltage from the 24V DC to power certain lower power circuit components. The pressure adjustment mechanism assembly 300 (shown in detail in FIG. 13) with its associated stepper motor 342, screw 342s, and nut 342n is also shown with respect to its placement in the console 30.

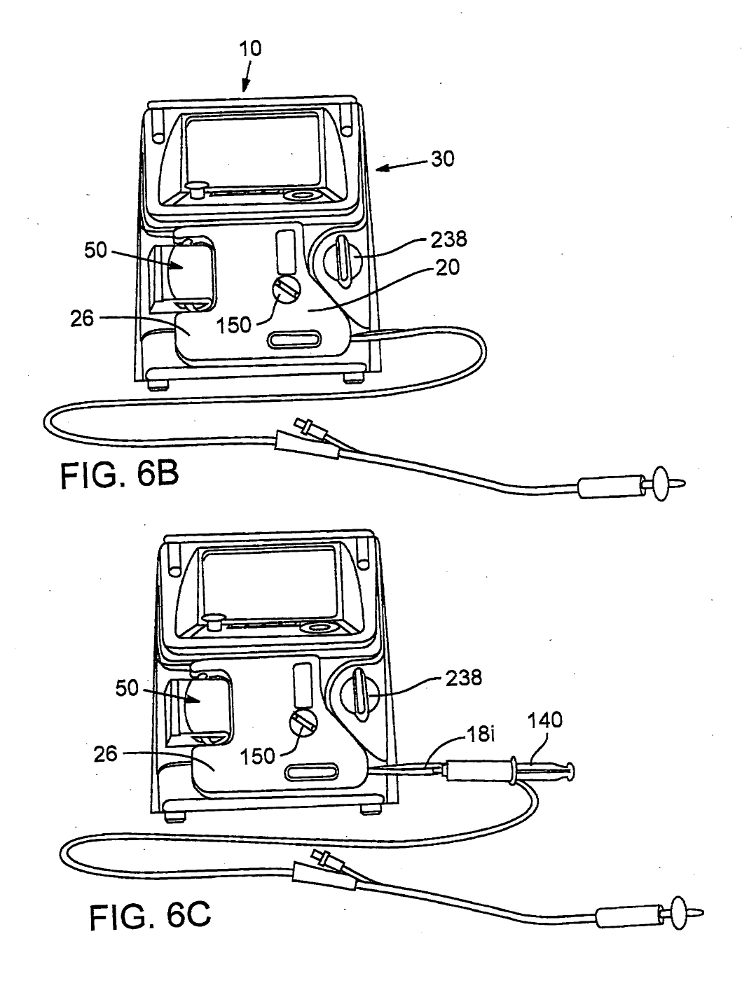

FIGS. 6A-6C illustrate a series of operational configurations according to embodiments of the present invention. As shown, the catheter assembly 20 with cassette 26 can be supplied separately and then mated to the console 30 at the use site. The console 30 is a multi-use device that can engage with any suitable catheter assembly 20 with a cassette 26 and any catheter configuration desired, the configuration will of course, correspond to the planned use in a desired region in the body. The pump 50 is configured to mate with the cassette 26 without requiring that an operator connect several loose hanging leads. As noted above, the conduit 24 can extend from an end region of the cassette 26. Of course, other conduit access configurations can also be employed as will be appreciated by those of skill in the art. FIG. 6B illustrates the cassette 26 mounted to the console 30. FIG. 6C illustrates the injection of a suitable amount of liquid into the flow path 18f and/or the syringe 40 using a syringe 140. In this embodiment, the liquid is inserted into a port 18i proximate the syringe 40 in a desired amount. Typically, the syringe 40 is filled to a level that is below capacity (about 50-75%) to allow for liquid removal during operation for pressure adjustment. Other liquid introduction means can also be employed. In certain embodiments, the internal syringe 40 can be pre-loaded with a desired quantity of liquid. The cassette-mounted syringe 40 can be in fluid communication with a liquid port 18i that can be formed as a valved T connection in fluid communication with the flow path 18f or other valve and port means to allow insertion of liquid and/or retention of the liquid in the system or syringe (not shown).

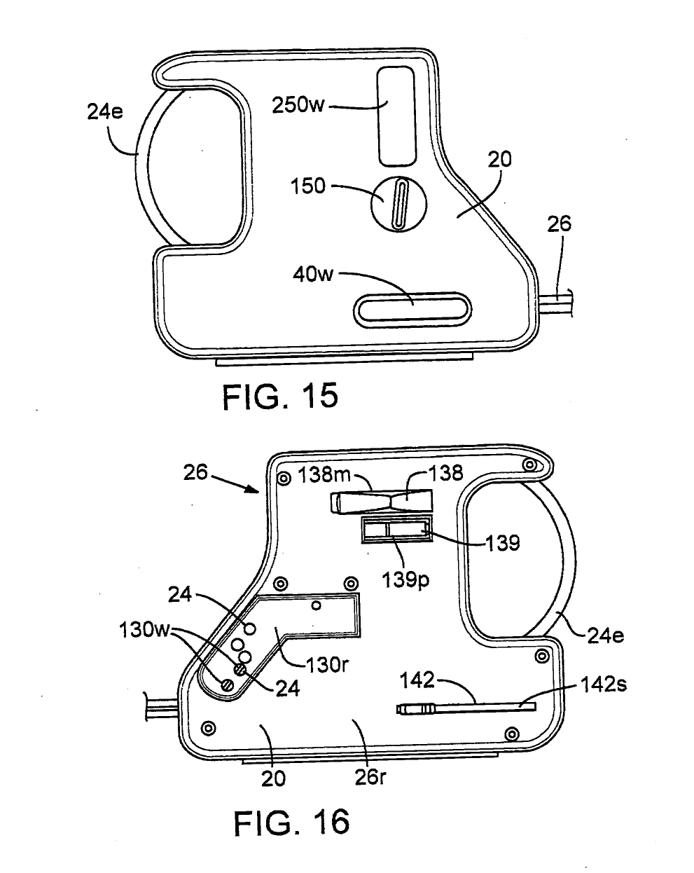

To load the primary or staring quantity of circulating liquid into the flow path 18f, a quantity of liquid is inserted into the flow path 18f. As shown in FIG. 17, the cassette 26 may include a pre-filled container 250 that is selectably able to be in fluid communication (or isolation) with the flow path 18f can be used and valves opened and closed via external knob or switch 150 (FIGS. 15, 17) located on the cassette 26. During initial start-up, the switch 150 can be turned to open the path to the container 250 and close the normal portion of the flow path that bypasses the container 250 (shown by arrows that go to and from container 50 and bypass a portion of the path 18f below the valve 150v in FIG. 17). The container 250 can be a flexible bag having about 50-200 ml of sterile water. As shown, the valve 150v can be formed by an elliptically shaped cam member 150c that rotates to force fingers 150f to expand outwardly away to compress the proximately positioned flow path shut against a stationary wall member and open the other flow path at desired times. Directing the liquid to travel through the container forces the air bubbles into the top portion of the bag 250 out of the liquid flow path 18f. The cassette 26 may be configured with a window 150w (FIG. 15) that allows a clinician to view the air bubble filtration.

FIGS. 7A and 7B illustrate the mating attachment of the cassette 26 to the console 30. As shown in FIG. 7A, the bottom portion of the cassette 26 is aligned with arm 42a and corresponding slot 42s (FIG. 3) and then the top portion of the cassette 26 can be pushed into position against the console mounting surface 30m. The knob 238 can be rotated to lock the cassette against the console 30.

FIG. 15 illustrates the front 26f of the cassette 26. As shown, the cassette 26 includes the switch 150 with control valve 150v (FIG. 17). The cassette 26 may also include a window over the container 250w and syringe 40w as desired.

FIG. 17 illustrates one embodiment of a cassette 26 encasing a portion of the conduit 24 forming the flow path 18f of a closed loop thermal treatment system 10. As shown, the cassette 20 includes a heater 14, a bi-directional (circulating liquid volume) pressure adjusting device 40, conduit that defines a portion of the circulating fluid flow path 18f, and a catheter 20 with an expandable treatment balloon 23. The circulating fluid flow path 18f includes a length of elastomeric conduit of tubing 18t extending between the catheter 20 and respective inlet and outlet portions of the circulation fluid flow path 18f. The arrows in the figure indicate the direction of the fluid flow through the system. The components can be arranged in different order and the liquid can flow in the reverse direction.

The heater 14 can be a tube through which the liquid flows and the liquid can be heated as it travels through the tube. The tube may be formed of a cylindrical silica, glass quartz, or ceramic configuration or other suitable material (such as metallic materials) through which the liquid flows can be heated. The cylindrical heater 14 may comprise a conductive vapor deposition film coating or other suitable conductive coating positioned over the outer surface of the cylinder that can be heated to heat the encased liquid flowing through the center of the heater. As shown in FIGS. 27A and 27B, and insulation sleeve 14s can be positioned over the heater 14 to help protect proximate components from undue exposure to heat during operation. In certain embodiments, the insulation sleeve 14s may be a fiberglass woven sleeve. Other embodiments include a cooling tube or bath (not shown). Other types of heating means may also be employed. A suitable cylindrical heating element is available from Thermostone, Inc., located in Marina, Calif. Another example of a conductive heating tube is described in U.S. patent application Ser. No. 09/433,952, the contents of which are hereby incorporated by reference as if recited in full herein.

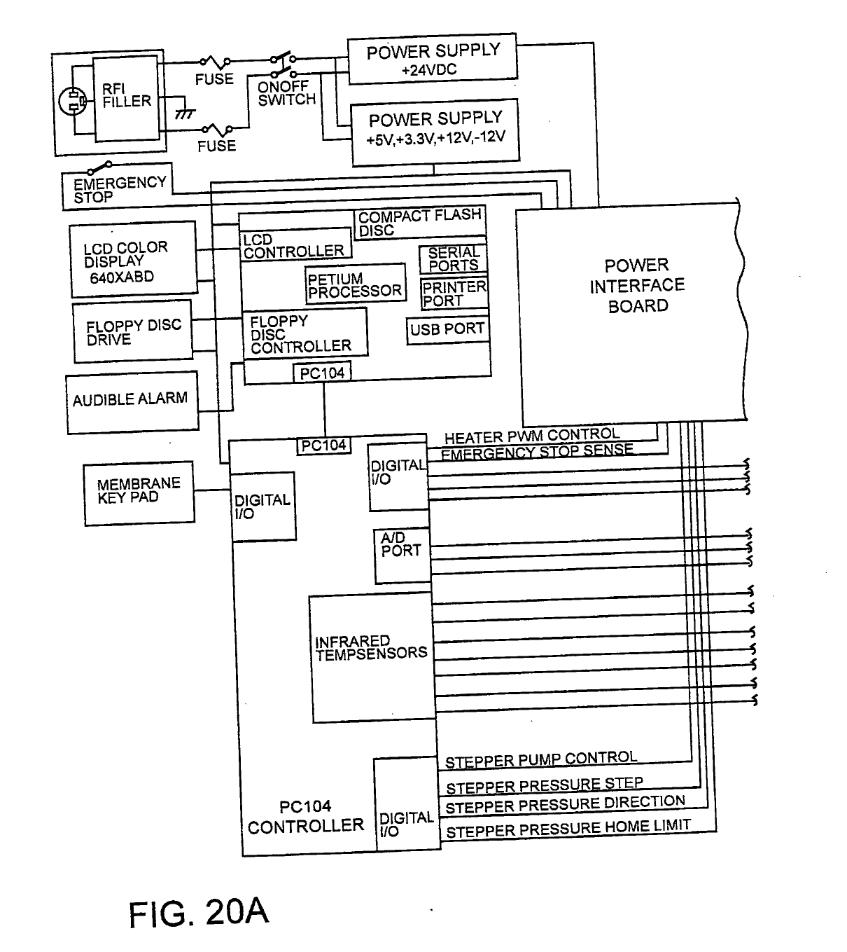

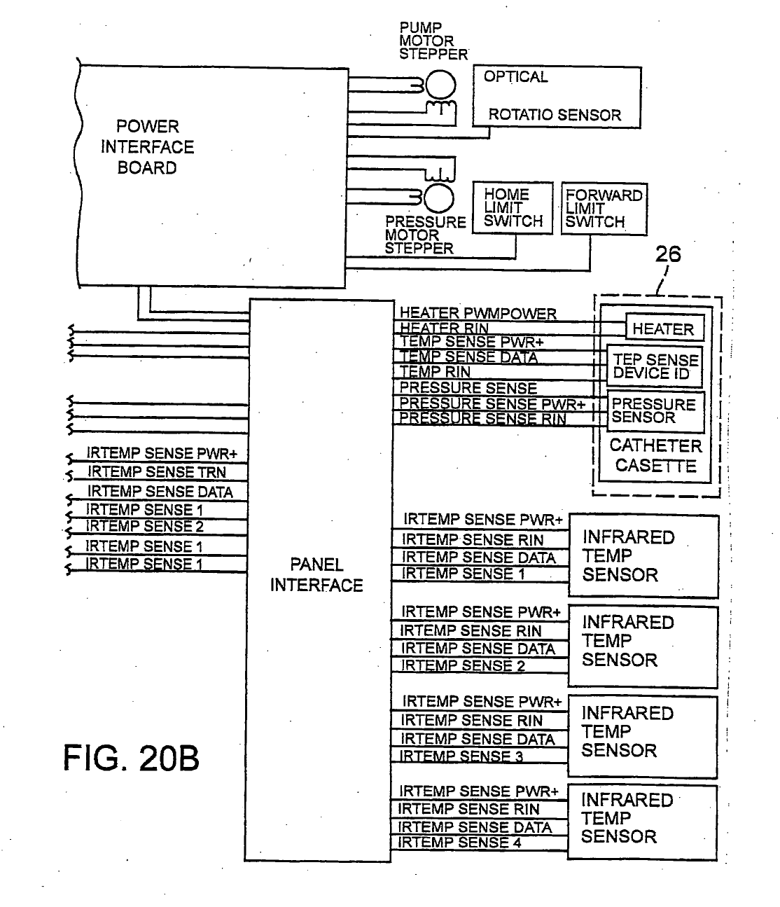

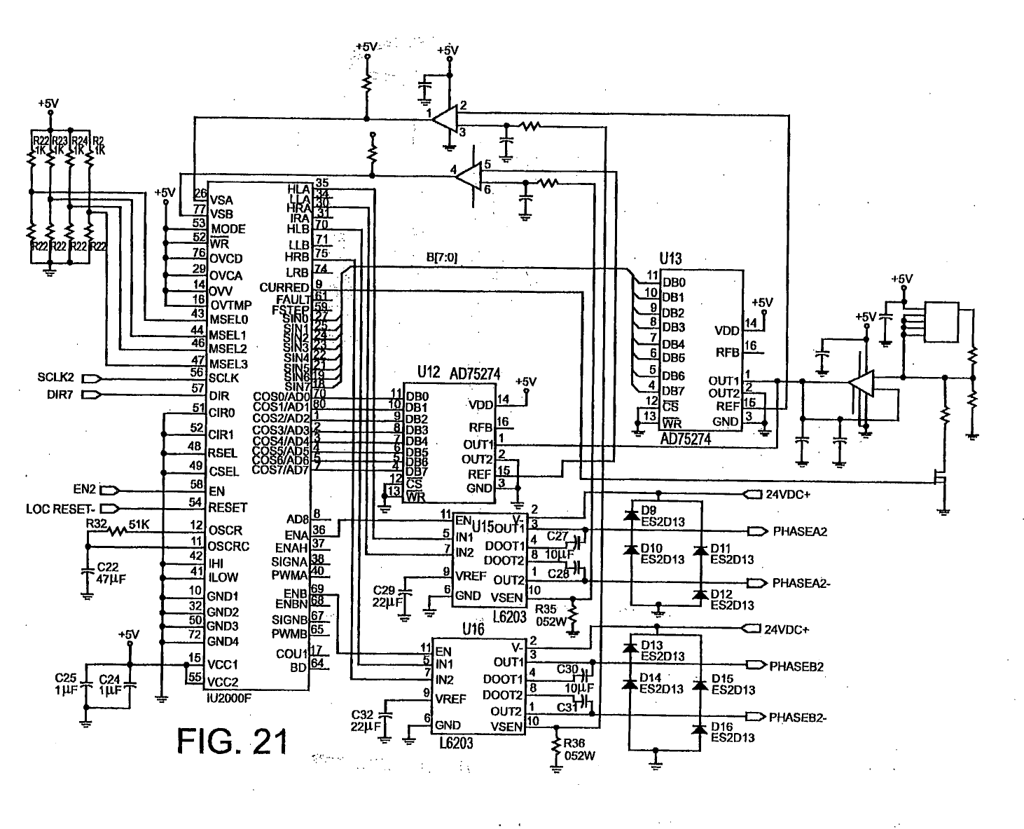

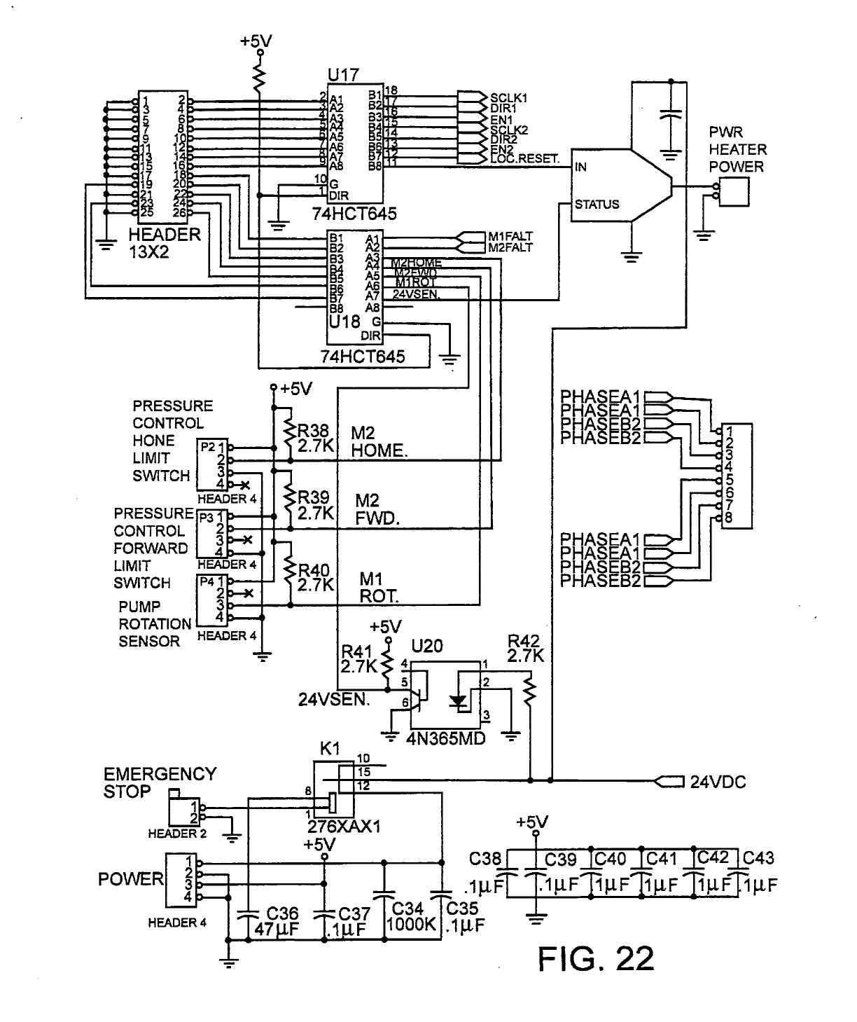

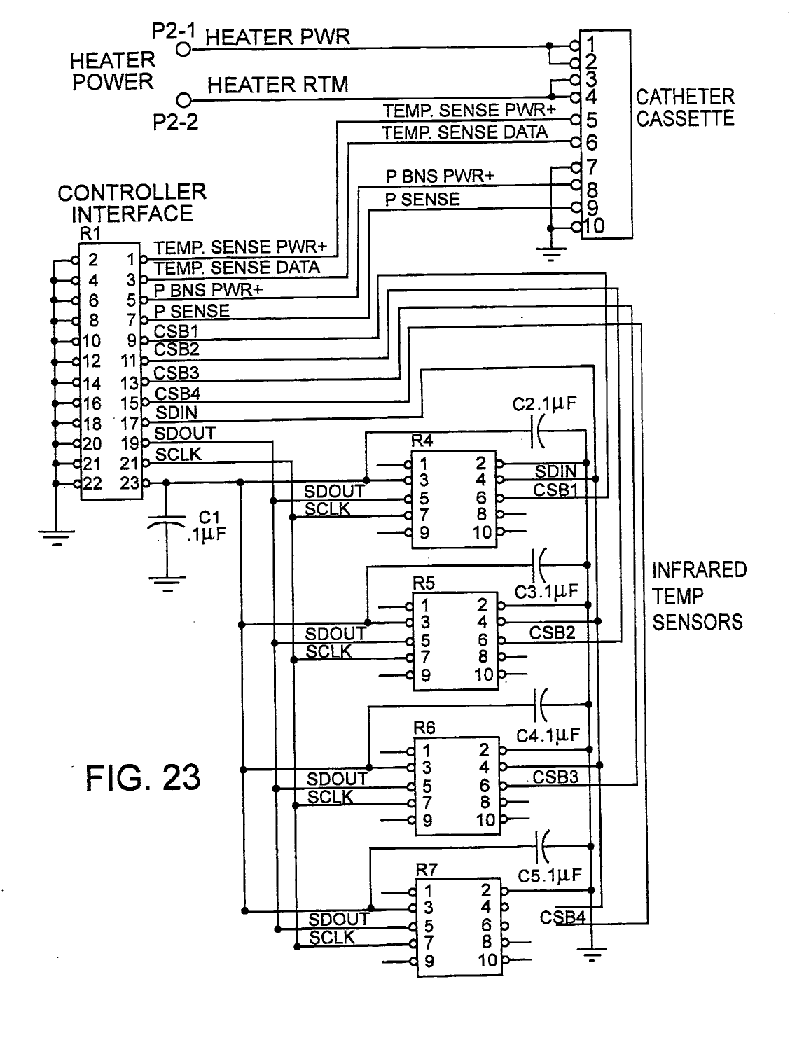

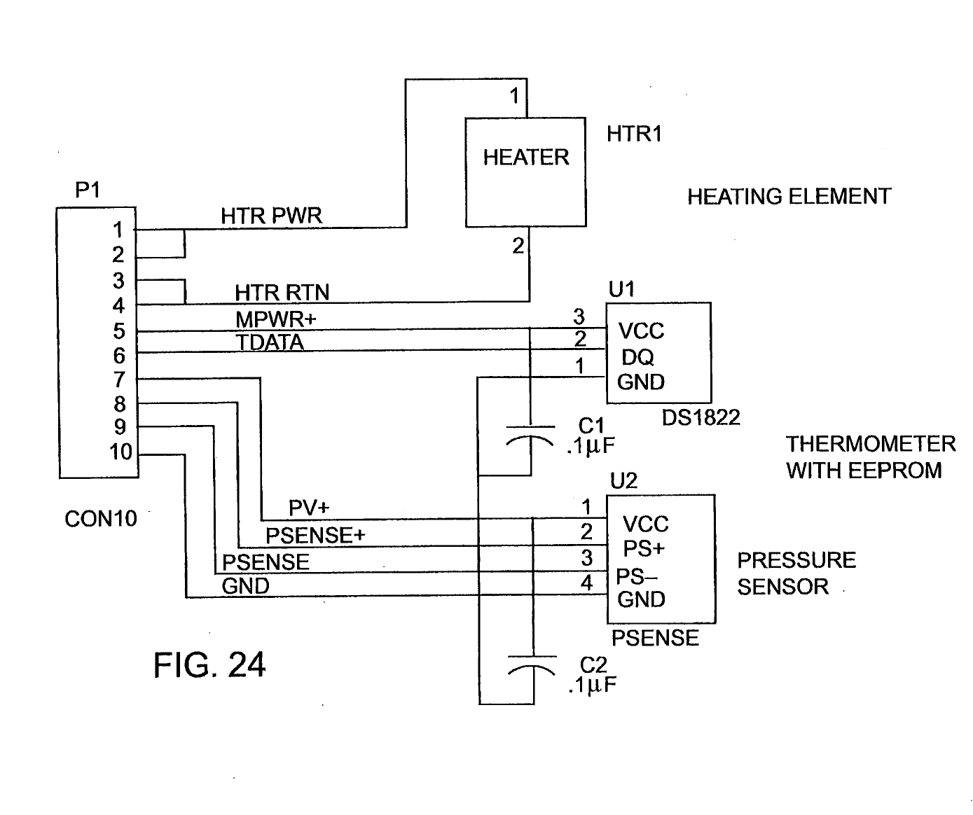

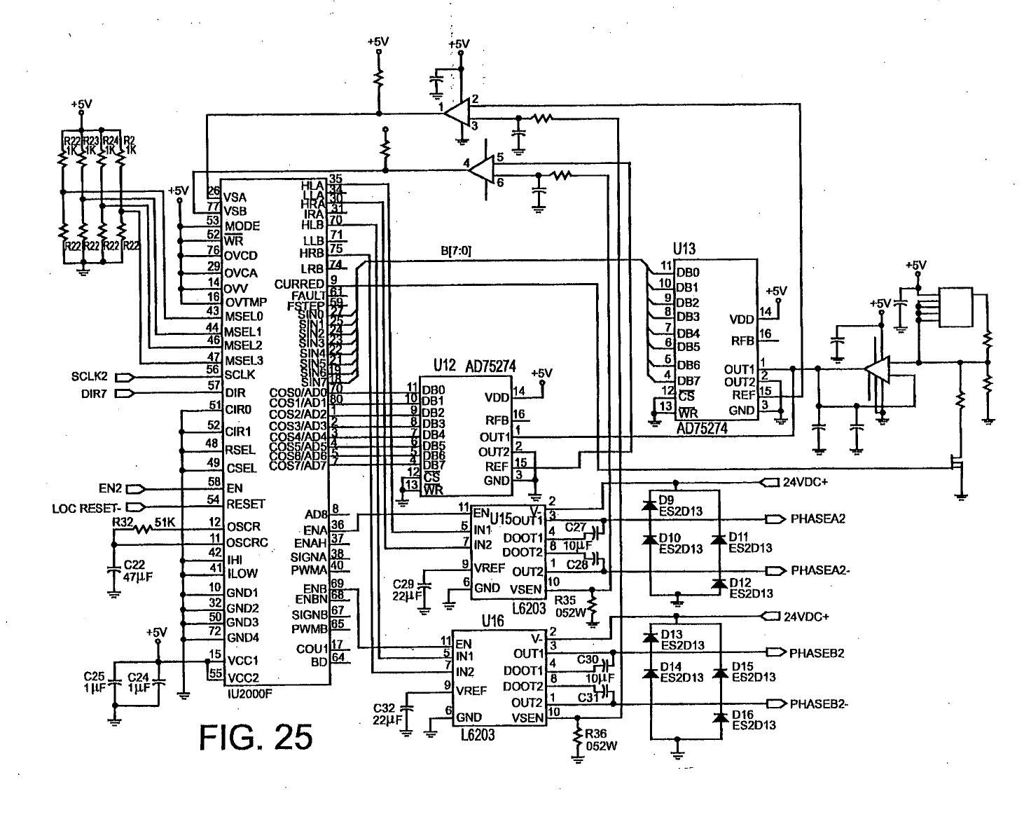

FIGS. 20A, 20B and 21-25 illustrate exemplary circuit schematics according to embodiments of the present invention. FIGS. 26A and 26B illustrate a circuit board 240 that includes a heat sensor 140 used as a re-use deterrent mechanism. In operation, the heat sensor 140 is located under the heater 14 and detects the heat and at the end of the thermal therapy the system 10 is configured to send a current spike to short out this component and render the cassette inoperable for re-use. The power connections to the console 30 are at the edge of the board in the location identified as to 139 (power interface on the cassette 26). An example of a suitable heat sensor 140 is a digital thermometer, such as model TO92 under part no. DS1822, produced by Dallas Semiconductor Corp., Dallas, Tex.

The system 10 may optionally include a user interface in communication with the controller to allow a user to adjust the pressure to a custom comfort level. This interface can be a joystick-type peripheral device, a touch screen on a display, a key input or membrane touch switch (such as an arrow) on a keypad, or a voice activated input (“raise” and “lower” or “pressure up” and “pressure down”), or other desired input means. The controller can include means to limit the pressure that the patient can introduce into the system (which may be combined with when the input can be operated), and thus, have a control override to a desired normal range of operation.

In the embodiment shown, the liquid is heated external of the subject (outside the body of the subject) and then introduced to the catheter. In certain embodiments, such as, but not limited to, BPH thermal ablation treatments, the circulating heated fluid can be introduced into the catheter at a temperature of about 45°-95° C. for a treatment period which is at least about 5-90 minutes in duration, and in particular embodiments heated to a temperature of between about 57°-62° C. for about 42-45 minutes in duration.

The system can be configured to provide economic, lightweight, portable circulating liquid closed loop thermal therapy systems with automated pressure monitoring and fine adjustment capability to be able to selectably control the pressure and temperature of the liquid in the closed loop system over the course of the treatment. The console can be configured as a vertical-rise housing with a reduced footprint to reduce the amount of square feet of real estate used in clinician or hospital facilities. In certain embodiments, the modular cassette is a substantially rigid body that is attached to the catheter and configured with a catheter repeat-use deterrent system (to promote hygienic sterile single uses).

The systems, methods, and devices can be configured with other enhanced operational features, including one or more of: (a) a plurality of pre-programmed selectable treatment procedures for different physiologic treatment conditions, each of the procedures can have different therapy times, temperatures, and pressures (in certain embodiments, each selectable procedure can have up to about five different steps, and the system can be pre-programmed with four different procedures); (b) automatic deflation of the treatment balloon to inhibit removal (and/or insertion) of the catheter when it is in an inflated state; (c) automatic smart chip or other repeat-use sensor recognition means, or automatic switch open/short at end of a procedure of the catheter assembly, to prevent or inhibit the re-use after the catheter assembly has been exposed to certain thermal thresholds; (d) infrared temperature sensors for the circulating fluid, (e) temperature safety override (optic coupling sensor for the pump motor and heating element sensors) as well as circulating fluid temperature sensor(s); (f) front panel mounted prominent emergency shutoff switch (g) light weight console (less than about 8.5 kilograms); (h) self-diagnosing trouble shooting program cycle; (i) accepts user input to accept patient identifier and automatically generates a patient record of treatment (electronic and/or paper form); (j) graphic display of status of treatment on monitor with two dynamic graphs of time and temperature; (k) downloadable treatment summary capability (local or remote monitoring of number and type of procedures run); (l) high speed pump-speed air purge cycle (typically at about 200 rpm or about twice the normal heating pump speed) to automatically prepare the system for circulating fluid without requiring hand pumping/manipulation of the treatment balloon; (m) disposable heating element in the cassette; (n) adjustable power input capacity having automatic recognition of 110 V 60 Hz or 220 50 HzAC cycle converted to 24VDC to power the system; (o) configured with separate chambers to facilitate electromagnetic shielding of graphic components from pump motor or power source; (p) orienting board component connectors on the same side in the console to be accessible via the back panel for easy field service access; (q) audible alerts at desired points in the treatment and/or for a malfunction; (r) easy external access port for software upgrades and/downloads; (s) environmental condition monitoring sensors (temperature/humidity); (t) back-up battery power supply to reduce operational disruptions; (u) surge protector to help maintain a level power supply input, and (v) pressure monitoring and control of the system during treatment.

The controller can have computer program code for (a) activating the pump, the heater, the temperature sensors, the pressure sensor and the pressure adjustment device to substantially continuously circulate heated liquid through the liquid circulation path at a controlled pressure and temperature; and (b) automatically adjusting the pressure in the liquid circulation path to compensate for operational pressure losses or to operate the system at a selected pressure at a desired time. The adjustment can be carried out to account for any physiological changes in the tissue proximate the targeted treatment region (such as in the prostatic urethra) so that the system maintains at least one selected operating pressure during administration of the thermal therapy. In certain embodiments, the system is configured to accept user input in situ to set the desired operating pressure(s), and other embodiments a series of increasing pressures are used to apply an increased pressure concurrently with heat at the target site in the body.

The controller can also have computer program code for: (a) disabling operating circuitry in the cassette after the treatment catheter has been used for one treatment procedure (making the cassette and catheter a disposable assembly to promote single-use catheters); (b) automatically deflating the treatment balloon at the end of the procedure before removal of the catheter (and/or at the beginning of the procedure post priming and before insertion into the subject); (c) activating a high speed air bubble purge procedure to filter air bubbles from the circulation path; (d) generating a patient record of treatment; (e) providing predetermined selectable treatment protocols; (f) performing a trouble shooting self-diagnostic test, and (g) automatically recording treatment parameters associated with the delivery of a patient's therapy.

As shown in FIG. 1, the treatment catheter 22 includes an anchoring balloon 22a, a treatment balloon 22b, and an elongated shaft 22s. The catheter 22 also includes inlet and outlet fluid circulating paths 24i, 24o, respectively, as well as a urinary drainage channel 28 (which can also be used to deliver medicaments therethrough while the catheter 22 is in position in the subject). The anchoring balloon 22a can be in fluid communication with the treatment balloon 22b, such that both are inflatable by the circulating heated fluid. Alternately, the anchoring balloon 22a can be fluidly isolated from the treatment balloon 22b (inflatable by a separate air channel directed thereto) (not shown). In this situation, the upper anchoring balloon 22a is separately inflatable and can be inflated before the treatment balloon 22b. This can reduce the likelihood that the upper balloon 22a will be inflated below the desired location (potentially introducing damage to the bladder neck or the upper portion of the prostate urethra) and facilitate proper positioning of the catheter 22 in the prostate relative to the bladder. The system 10 can be configured to resist disconnection or to impede the withdrawal of the catheter from the subject until the pressures in the anchoring balloon 22a and the treatment balloon 22b indicate a deflated state. Other catheter configurations can also be used as noted above (including those for sized and configured for arteial uses, female urinary, urethra, endometrium, uterine, or other body lumens, or cavities. See, e.g., U.S. Pat. No. 5,084,044, the contents of which are hereby incorporated by reference as if recited in fill herein.

It is noted that the circulating heated fluid for thermal ablation treatments can be heated to temperatures above about 45° C. and delivered to the targeted tissue to provide the thermal temperatures for different applications for different lengths of treatment as the desired application dictates. For example, this can be carried out by heating the circulating temperature to at least about 50° C. and then circulating the heated liquid into the catheter, which is positioned in the desired location in the subject so as to expose the targeted tissue to the heated circulating temperature for about 5-90 minutes, and typically about 20-45 or 20-60 minutes.

A suitable thermal treatment system and treatment catheters are available from ArgoMed, Inc. located in Cary, N.C. See also, U.S. Pat. Nos. 5,257,977 and 5,549,559 to Eshel, and co-assigned U.S. patent application Ser. No. 09/433,952 to Eshel et al., the contents of which are hereby incorporated by reference as if recited in full herein.

The catheter 22 can include a region with increased insulation 29 with respect to other portions of the catheter so as to protect non-targeted tissue from exposure to the circulating heated liquid. The insulated regions 29 can be configured on the catheter as an extra layer or thickness of a material along the proximal or lower shaft portion. Other treatment catheters include a series of circumferentially arranged elongated air channels or conduits which encircle the heated circulating fluid passages and provide thermal insulation along the elongated shaft portion of the catheter as described in U.S. Pat. Nos. 5,257,977 and 5,549,559 to Eshel, the contents of which are hereby incorporated by reference as if recited in full herein. See also, co-pending and co-assigned U.S. patent Ser. No. 10/011,700, for additional description of suitable catheters, the contents of which are also incorporated by reference as if recited in full herein.

FIG. 17 illustrates a pressure adjustment device in communication with a pressure sensor 15s in the closed loop system 10. As shown, the pressure sensor 15s can be located in the cassette 26 external of the body and away from the catheter 20. The pressure adjustment device can be arranged such that it is in-line or offset from the liquid circulation path 18f. The travel distance of the circulating liquid can be from about 10-20 feet or more, and is typically about 14-16 feet.

In operation, fluid, which can be water or a water-based liquid, can be heated external of the subject, directed into the catheter 20, and circulated in the enclosed fluid paths 24i, 24o in the catheter 22. The liquid is directed through the shaft 22s via the inlet path 24i to the treatment balloon 22b located proximate the desired treatment site, out of the treatment balloon 22b to the outlet path 24o, and out of the subject. The circulating fluid is directed into the treatment balloon 22b, which then expands in response to the quantity of fluid held therein. As shown, infrared or other suitable temperature sensors 124 can be configured so that one is in communication with the inlet portion or side of the path 18f (upstream of the catheter), and the other on the outlet portion or downstream side of the path 18f can be used to control the temperature of the circulating liquid.

A low volume of liquid, meaning below about 100 ml (that in certain embodiments can be below about 50 ml, or even in particular embodiments, below about 20 ml) of circulating heated liquid is physically circulated, during operation, at least initially, through the circulation path 18f of the closed loop system 10 to deliver the thermal (or thermal ablation) treatment via the treatment catheter 22. In certain embodiments, water that has been sterilized, distilled can be used as the circulating liquid medium.

The circulating fluid (and the anchoring balloon inflation media, when separately inflatable) is preferably selected to be non-toxic and to reduce any potential noxious effect to the subject should a situation arise where the balloon integrity may be compromised, accidentally rupture, leak, or otherwise become impaired during service.

The catheter 22 can be flexibly configured so as to be able to bend and flex to follow the shape of the lumen or cavity as it is introduced into the lumen or cavity until a distal portion of the catheter 22 reaches the desired treatment site.

The catheter 22 can be sized as an elongated tubular body with a relatively small cross-sectional area having a thin outer wall so as to be able to be inserted into and extend along a length of the desired lumen to reach the desired treatment site. As used herein, the term “thin outer wall” means a wall having a thickness of about 2 mm or less, and preferably about 1.2 mm or less, and can be in certain embodiments about 0.5 mm or less. For prostate or male urinary applications, the cross-sectional width or outer diameter of the catheter 22 about the tubular body is preferably between about 6-8 mm (18-24 French). Of course, as noted above, the flexible catheter 22 can be alternatively sized and dimensioned to fit other lumens, cavities and/or treatment applications.

In certain embodiments, a major portion of the cross-sectional area of the shaft region 22s of the catheter 22 is taken up by the size of the fluid channel, or channels, held therein. In certain embodiments, such as, but not limited to, those directed to prostate or male urinary applications, the catheter 22 can include at least three separate fluid channels: the circulating inlet and outlet channels 24i, 24o and the fluid drainage or medicament delivery channel 28 in the shaft region 22s.

The flexible catheter 22 can also be configured such that it is sufficiently rigid to be able to maintain an opening in the drainage lumen 28 when inserted and in position in situ (and exposed to increased system pressures of about 0.5-3 atm, and typically at least about 1-2 atm during at least a portion of the thermal therapy) so that the catheter is configured to retain at least about 50% of the cross-sectional area, and preferably at least about 75%-90% or more, of the cross-sectional area, of the drainage lumen 28 relative to the pre-insertion catheter size. As such, the catheter 22 can be flexibly configured such that it is sufficiently conformable to yield to the contours of the subject's body as it is inserted therethrough and into position in the desired region of the subject, yet sufficiently rigid to provide an open drainage lumen when it resides in position in the body (such as in the prostate), and exposed to tissue which is exhibiting distress during or subsequent to undergoing a therapy or thermal treatment.

In certain embodiments, the catheter 22 can be configured such that it is able to maintain a sufficiently sized drainage opening in the drainage lumen 28 to allow desired flow volumes therethrough when exposed to compressive pressures from the treated tissue on the order of about 0.5 atm (7 psi)-2 atm (28 psi) or 3 atm (42 psi) after exposure to elevated temperatures above about 45° C. for at least about 5-10 minutes, and more preferably for above about 20-30 minutes. The catheters 22 of the instant invention can also be used to maintain an open passage of desired size for other treatments or applications where there is a desire to maintain the open passage in a flexible catheter which is exposed to edema or stress in the subject See co-pending and co-assigned U.S. patent application Ser. No. 10/011,700 for additional description of suitable catheter configurations, the contents of which are hereby incorporated by reference as if recited in full herein.

FIG. 17 illustrates that the system 10 includes at least one pressure sensor 15s in communication with the pressure-adjusting device that is configured to adjust the system pressure responsive to the detected pressure during the delivery of the thermal therapy. The sensor 15s may be positioned in a number of locations along the fluid or liquid circulation path 18f. As shown, the sensor 15s can be located on the system 10 such that it is outside the body of the subject or patient during operation and able to detect system operating pressures which are representative of the pressure at the treatment balloon, as the treatment balloon defines a portion of the liquid circulation path. The pressure adjustment device may be any suitable mechanism, an exemplary embodiment using a syringe and stepper motor will be discussed further below.

The pressure sensors 15s can be of any suitable type, such as, but not limited to, transducers similar to those used to measure blood pressure and digital pressure gages. Examples of pressure sensors include the MERITRANS transducer from Merit Medical Systems of South Jordan, Utah, the Medex (MX960) transducer from Medex of Dublin, Ohio, and the Digibar II, PE300, digital pressure gage from HBM GmbH (Hottinger Baldwin Messtechnik) of Germany and similar device identified as Model No. DPG1000L-30G from Omega, of Engineering, Inc., of Stamford, Conn. with a pressure range of 0-30 psi and temperature range of 0-70° C.

In certain embodiments, the tubing 24 can have an inner diameter of about 2-20 mm, and typically about 2.5 mm The Merit and Medex transducers are rated for a compensated pressure range of −10 to 300 mm Hg (maximum design pressure of about 5 psi) and may be used to measure up to about 20 psi in the system. Another sensor 15s type is a digital pressure gage with a digital readout in bars (the HBM model as noted above). The gage can be mounted off of a “T” connection with the tubing 24. The T connection can be sized so as not to constrict flow with openings larger than the (2.5 mm) inner diameter of the tubing.

Referring now to FIG. 17, one embodiment of a pressure adjustment device is shown. This embodiment employs a syringe 40 with a quantity of liquid held therein. A plunger or piston 40p is used to direct fluid out of or into the syringe 40 from a supplemental fluid path 18s. As shown, a Y connector 72 defines a junction between the liquid circulation path 18f and the supplemental fluid (adding and removing) path 18s. Other connector or joint types can also be used (such as T's or other configurations). To increase the pressure in the system, additional liquid is injected into the circulation path. Similarly, to decrease the pressure in the system, the liquid can be directed back into the syringe 40. The Y connector 72 can be positioned upstream of the catheter inlet 24i in the cassette such that the syringe 40 is in fluid communication with the liquid circulation path 18f. In certain particular embodiments, the Y connector 72 and syringe 40 are located downstream of the heater 14 and upstream of the catheter inlet in the cassette 26. In certain embodiments, the syringe 40 can be configured to hold between about 30-100 ml, and typically between about 30-50 ml of liquid. The handle or arm of the syringe plunger 40p can be connected to a stepper motor assembly 300 (FIG. 13) which can direct the controlled translation of the plunger and the injection or removal of liquid from the liquid circulation path 18f to maintain or adjust the system 10 to the desired operational pressure. Other suitable control mechanisms can also be used as will be appreciated by those of skill in the art.

The systems or methods may be used to treat BPH, prostatitis, or other urinary or body conditions. For BPH applications, the liquid can be heated external of the body to a temperature in the range of between about 57°-62° C. or greater. The circulating heated liquid is directed through the catheter to a treatment balloon such that it travels, captured in the catheter, through the penile meatus, along the penile urethra the bulbous urethra, and the membranous urethra to a localized treatment region in the prostate. The tissue in the localized treatment region in the prostate is exposed to a temperature above about 45° C. for a predetermined thermal ablation treatment period by exposure to the conductive heat from the heated circulating liquid (the liquid can be input at or above about 60° C. for more than about 5-30 minutes, and typically for about 37 minutes). As noted above, the localized treatment region can be the prostatic urethra, leaving the membranous urethra (and the sphincter and penile meatus), non-ablated. This is accomplished in circulating systems (which heat remotely) by insulating the shaft of the treatment catheter up to the treatment balloon to inhibit the exposure of non-targeted tissue to ablation temperatures. Thus, in certain embodiments, the non-targeted tissue is insulated so that it is exposed to a maximum temperature of below about 45° C. from contact with the treatment catheter during the thermal therapy. Additionally, the catheter can be configured to allow urine to drain through the treatment catheter during the procedure.

The thermal therapy can be carried out to increase or maintain the system operating pressure over time (and temperatures can be increased and decreased during the treatment as well as desired). The pressure can be held substantially constant or above certain threshold pressures during a major portion of the ablation treatment time so that the patient is exposed to pressures between about 0.75-2 or 3 atm (which can be carried out with concurrent exposure to ablation temperatures of between about 45°-95° C.).

The pressure can be held substantially constant (and elevated) during substantially the entire thermal treatment. The pressure can be gradually increased in a linear manner over the thermal treatment (so that the end of the treatment employs a higher pressure relative to the beginning of the treatment). The pressure can be increased more rapidly during an initial portion of the therapy and then increased more gradually (or held substantially constant) toward the end or a latter portion of the treatment.

Two or more sequential treatment periods with different temperatures and/or pressures can be used. The initial pressure can be less than the next or a second pressure during a second subsequent portion of the treatment (T2), the second pressure can be maintained for a longer portion of the treatment. The first or initial pressure can be concurrently applied to the subject with heat supplied at an initial temperature that is less than a subsequent or second temperature. In certain embodiments, a first lower temperature/lower pressure combination can be used until the subject develops less sensitivity to the treatment (typically after exposed nerves are killed at about 5-10 minutes into the treatment).

The initial pressure can be increased during T1 and held substantially constant during T2 and then increased again during T3 and then held substantially constant during T4 such that the latter portion of the treatment is carried out at higher system pressures than the prior portions. For example, the following sequence of pressures can be used: an initial pressure of about 0.3-0.5 atm ramped over T1 (about 5-10 minutes) to about 0.5-1 atm where it is held during T2 (5-10 minutes), then ramped again during T3 to about 1-2 atm (for about 5-10 minutes), and then held at about 1-2 atm for T4 (about 5-30 minutes). The series of sequentially increasing pressures can be used to deliver the thermal therapy. Alternatively, selected ones of these can be either held substantially constant during that portion of the treatment or gradually ramped or increased during the therapy. This increased pressure can enhance the depth of penetration into the tissue. Setting pressures to predetermined levels can make the treatments more consistent patient to patient irrespective of the physiology of the prostate or the length of the treatment balloon or other variables in the system.

The patient may control the pressures during substantially the entire active thermal treatment (T) or at selected portions of the treatment. For example, at an initial T1, or subsequent portion of the treatment, T1. Patient to patient, the pressure increments may vary depending on the patient's tolerance for pain (shown by the different pressure lines, numbered as “1” and “2”). It is contemplated that when a patient has some control over the procedure, he may be more apt to select or willing to experience greater pressures. The system can be programmed with a safety override that prevents over-pressures from being selected (shown by the upper limit in the figure). In addition, a lower limit can be set so that the patient cannot select non-suitable operating conditions (not shown). The upper and lower limits may be a constant value or can be altered depending on the duration or point in time in the treatment (not shown).

Table 1 provides examples of pressures and temperatures.

TABLE 1

| P1 | T1 | P2 | T2 | P3 | T3 | P4 | T4 |
|---|---|---|---|---|---|---|---|
| 0.3-1 atm | 40-55° C. | 1-3 atm | 45-95° C. | n/.a | n/a | N/a | n/a |
| 0.5-1 atm | 45-50° C. | 1-2 atm | >57-62° C. | n/a | n/a | N/a | n/a |
| 0.5-3 atm | 40-44° C. | n/a | n/a | n/a | n/a | N/a | n/a |
| 0.3-1 atm | 40-50° C. | 0.5-1 atm | 40-57° C. | 1-3 atm | 40-95° C. | 1.5-3 atm | 40-95° C. |

For decreasing pressures during the thermal ablation treatment, the penetration depth may be reached at times below about 20 minutes into the treatment (before T1). The latter portion of the ablation treatment may promote body reaction or response to heat damage of the tissue (edema), but may not significantly impact on the depth of penetration into the tissue. That is, about 80-90% of the tissue penetration may occur during the first 10-20 minutes. The ablation temperatures may be between about 57°-62° C. or greater (typically below about 95° C.).

It will be understood that certain of the features, operations, or steps described above may be implemented or directed to be carried out by computer program instructions. Accordingly, as will be appreciated by one of skill in the art, the present invention may be embodied as a method, data processing system, or computer program product. Accordingly, the present invention may take the form of an entirely hardware embodiment, an entirely software embodiment or an embodiment combining software and hardware aspects all generally referred to herein as a “circuit.” Furthermore, the present invention may take the form of a computer program product on a computer-usable storage medium having computer-usable program code means embodied in the medium. Any suitable computer readable medium may be utilized including hard disks, CD-ROMs, optical storage devices, a transmission media such as those supporting the Internet or an intranet, or magnetic storage devices.

Computer program code for carrying out operations of the present invention may be written in an object oriented programming language such as Java®, Smalltalk or C++. However, the computer program code for carrying out operations of the present invention may also be written in conventional procedural programming languages, such as the “C” programming language. The program code may execute entirely on the modular unit as a stand-alone software package, partly on a user's (clinician's) computer and/or partly on a remote computer, or entirely on the remote computer. In the latter scenario, the remote computer may be connected to the modular unit and/or user's computer through a local area network (LAN) or a wide area network (WAN), or the connection may be made to an external computer (for example, through the Internet using an Internet Service Provider).

The present invention is described with reference to illustrations and/or description of operations of apparatus (systems), circuits, and computer program products according to embodiments of the invention. It will be understood that selected features or operations in the illustrations and/or description, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions specified.

These computer program instructions may also be stored in a computer-readable memory that can direct a computer or other programmable data processing apparatus to function in a particular manner, such that the instructions stored in the computer-readable memory produce an article of manufacture including instruction means which implement the function or functions specified.

The computer program instructions may also be loaded onto a computer or other programmable data processing apparatus to define a device and/or cause a series of operational steps to be performed on the computer or other programmable apparatus to produce a computer implemented process such that the instructions, which execute on the computer or other programmable apparatus, provide steps for implementing the functions specified. These computer program instructions may also be stored in a computer-readable memory that can direct a computer or other programmable data processing apparatus or associated hardware equipment to function in a particular manner.

In certain embodiments, the system controller or other operably associated computer device can include computer program code for: (a) activating the pump, the heater, the temperature sensor(s), the pressure sensor and the pressure adjustment device to substantially continuously circulate heated liquid through the liquid circulation path, and (b) automatically adjusting the temperature to desired operational temperatures and automatically adjusting the pressure in the liquid circulation path to compensate for operational pressure losses in the treatment system over a treatment time of at least about 5 minutes and to account for any physiological changes in the tissue proximate the targeted treatment region in the prostatic urethra so that the system maintains at least one selected operating pressure during administration of the thermal therapy.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

That which is claimed is:

1. An ambulatory thermal treatment system having a closed loop liquid circulation path configured to administer a thermal treatment to a patient, comprising:
(a) a portable control console configured to control the delivery of a thermal treatment to a patient, the console having a cassette mounting region, the console comprising:
a power supply;
a controller operably associated with the power supply;
a pump operably associated with the controller, wherein, in operation, the pump is configured to circulate liquid through a closed loop circulation path to administer a desired thermal therapy to a patient;
(b) a cassette sized and configured to be releasably mounted to the console at the console mounting region, wherein the cassette houses a portion of the closed loop circulation path; and
(c) a graphic display disposed on the front portion of the console, wherein the system is configured to substantially continuously circulate a low volume of liquid in the closed loop circulation path during operation.

2. A system according to claim 1, further comprising a length of flexible conduit a portion of which is held in the cassette, the length of conduit forming a portion of the closed loop circulation path, wherein a portion of the conduit held in the cassette is configured so as to be in communication with the pump when the cassette is mounted to the console.

3. A system according to claim 1, wherein the cassette has a substantially rigid body with a perimeter boundary, and wherein the conduit is held in the cassette so that an intermediate portion of the flexible conduit in the cassette defines a portion of the closed loop circulation path that is configured to serially exit the perimeter boundary of the cassette and extend outwardly a distance beyond the perimeter of the cassette to provide an externally accessible segment, then re-enter the perimeter boundary of the cassette.

4. A system according to claim 3, wherein the length of flexible conduit includes an inlet conduit and an outlet conduit for circulating liquid in a closed loop flow path, said system further comprising a treatment catheter having a circulating liquid inlet channel, a circulating liquid outlet channel, and an expandable treatment balloon in fluid communication with the catheter inlet and outlet channels, wherein the length of flexible conduit and the catheter define the closed loop circulation flow path with the treatment catheter inlet channel being configured to attach to said inlet conduit and the treatment catheter outlet channel being configured to attach to said outlet conduit so as to be able to circulate fluid in the closed loop circulation flow path.

5. A system according to claim 4, further comprising:
a quantity of liquid in the circulation flow path;
a heater held in the cassette operably associated with the liquid in the circulation flow path;
a pressure sensor operably associated with the liquid in the circulation flow path mounted in the cassette; and
a pressure adjustment device operably associated with the pressure sensor in the cassette, the system circulation flow path, and the controller in the console.

6. A system according to claim 5, wherein the console controller comprises computer program code for:
(a) activating the pump, the heater, the pressure sensor and the pressure adjustment device to substantially continuously circulated heated liquid at a predetermined temperature through the circulation flow path during operation;
(b) automatically adjusting the pressure in the circulation flow path to a pre-determined pressure level so that the system maintains at least one selected operating pressure during operation; and
(c) accepting user input for selecting the desired thermal therapy.

7. A system according to claim 6, wherein the computer code further comprises code for adjusting the operational pressure in the circulation flow path to a predetermined constant and/or adjustable pressures associated with the selected thermal therapy.

8. A system according to claim 1, further comprising means for automatically deactivating/and or destroying an operating component in the cassette at the end of a thermal procedure thereby deterring re-use of the cassette.

9. A system according to claim 2, wherein the console comprises a plurality of infrared sensors configured to be in optical communication with the conduit in the cassette when the cassette is mounted to the console.

10. A system according to claim 4, wherein the catheter and the cassette are configured as single-use disposable devices.

11. A system according to claim 1, wherein the cassette is a substantially rigid body that releaseably locks to the console during operation.

12. A system according to claim 4 wherein the console controller has computer program code for timing the duration of the treatment administered by the system and directing the automatic deflation of the treatment balloon at the end of a thermal therapy before a clinician removes the catheter from the body of the subject.

13. A system according to claim 5, wherein the console controller comprises computer program code for monitoring the operation of the pump and heater to generate an audible alarm when abnormal activity is detected.

14. A system according to claim 3, wherein the console includes opposing front and rear portions, wherein the externally accessible conduit segment in the cassette extends outwardly from the cassette body with a closed curvilinear profile, the curvilinear shape sized and configured to engage with the pump on the front portion of the console, and wherein the console comprises a sensor for detecting when the externally accessible conduit Segment extending from the cassette is improperly aligned with the pump.

15. A closed loop circulating liquid thermal treatment apparatus, comprising:
a portable console having a mounting surface with a power interface region thereon, the console comprising a controller, pump, and power supply therein, the power supply being in electrical communication with the power interface region on the console; and
a cassette housing having opposing front and rear primary surfaces, the rear surface including a power interface region, wherein the cassette housing is sized and configured to releasably mount to the mounting surface of the console so that the cassette power interface region engages with the console power interface region to be in communication with the controller and power supply in the console, and wherein the cassette holds a
heating element therein, wherein, in use, the operation of the heating element is controlled by the controller in the console.

16. A system according to claim 15, further comprising a length of flexible conduit held in the cassette, the flexible conduit defining a portion of an enclosed circulating liquid flow path.

17. A system according to claim 16, wherein an intermediate segment of the flexible conduit held in the cassette is configured to releaseably engage with the pump when the cassette is mounted to the console.

18. A system according to claim 17, wherein the cassette has a substantially rind body with opposing upper and lower arm portions, wherein the intermediate segment of the flexible conduit is configured with a curvilinear intermediate segment that extends outside the bounds of the rigid cassette body so that one end portion of the curvilinear segment exits the upper arm portion mud the opposing end portion of the curvilinear segment enters the lower arm portion.

19. A system according to claim 17, further comprising a treatment catheter having an expandable treatment balloon thereon, the treatment catheter being configured for insertion into a body cavity or natural lumen 6f a subject, wherein the treatment catheter has separate inlet and outlet circulation channels configured to circulate liquid to and from the treatment balloon, respectively, and wherein the treatment catheter is configured to be operably associated with the cassette and in fluid communication with the conduit during use.

20. A system according to claim 16, wherein the console comprises a locking member that secures the cassette to the console during operation to inhibit inadvertent removal during administration of a thermal treatment procedure.

21. A system according to claim 16, further comprising means for automatically destroying circuit components in the cassette after completion of a treatment procedure to inhibit re-use of the cassette.

22. A single-use disposable cassette for a thermal treatment system, comprising:
a cassette housing configured with opposing front and rear surfaces, wherein the cassette is sized and configured to be releasably mounted to a control console that controls the administration of a thermal therapy to a subject;
a pressure sensor held in the cassette;
a cylindrical heater having a central liquid flow channel therethrough held in the cassette;
a temperature sensor operably associated with the heater held in the cassette;
an externally accessible power interface connection operably associated with the heater, temperature sensor, and pressure sensor; and
a length of flexible conduit held in the cassette defining a portion of a circulating liquid flow path, a portion of the length of flexible conduit in the cassette being in communication with the heater.

23. A cassette according to claim 22, further comprising an insulating sleeve positioned over the heater in the cassette.

24. A single-use disposable catheter assembly, comprising:
a modular cassette housing having opposing front and rear surfaces, the cassette housing a portion of a circulating liquid flow path, the front surface comprising:
an air bubble filtration button that selectably controllably alters the configuration of the circulation flow path of liquid flowing through the cassette;
a pressure sensor held in the cassette so that it is in communication with the circulating liquid flow-path;
a pressure adjustment syringe having an associated plunger held in the cassette so that syringe is in fluid communication with the circulating liquid flow path;
at least one infrared sensor window formed in the rear surface of the cassette housing;
a pressure adjustment slot formed in the rear surface of the cassette housing proximate the pressure adjustment syringe, wherein, in operational position, the slot is adapted to receive a translating member therein that advances and retracts the plunger of the syringe; and
a treatment catheter configured for insertion into the natural lumen or body cavity of a subject, the treatment catheter having an expandable treatment balloon thereon, wherein the treatment catheter defines another portion of the circulating liquid flow path and is in communication with the portion of the liquid circulating flow path held in the cassette.

25. A catheter assembly according to claim 24, further comprising an insulating sleeve positioned over the heater.

26. A catheter according to claim 25, wherein the sleeve comprises a woven fiberglass material.

27. A method of treating a subject using a closed loop thermal treatment system haying a console configured to receive and secure a modular cassette member thereon, comprising:
providing a portable console of a thermal treatment system having a power source, a pump, and electronic circuitry therein;
mounting a modular cassette member having a length of flexible conduit onto the console so that a portion of the length of flexible conduit engages with the pump in the console;
securing the cassette to the console;
accepting user input to select one of a plurality of different types of pre-programmed thermal therapy procedures; and
removing the cassette from the console after termination of the selected thermal therapy procedure.

28. A method according to claim 27, further comprising serially repeating the steps of mounting, securing, and accepting for each patient undergoing a thermal therapy treatment.

29. A method according to claim 27, further comprising automatically disabling at least one selected component in the cassette so that operational circuitry associated therewith is inoperative to deter re-use of the cassette at a desired time during or after the administration of the selected thermal therapy while the cassette is mounted to the console.

30. A method according to claim 29, further comprising circulating liquid in a flow path defined by a closed loop flow path extending through flexible conduit in fluid communication with the flexible conduit in the cassette and a treatment catheter.

31. A method according to claim 29, further comprising inserting a treatment catheter into a subject and administering the selected thermal therapy thereto before the disabling step is carried out.

32. A method according to claim 31, wherein the inserting step is carried out by:
inserting a treatment catheter having a liquid circulation path and an expandable treatment balloon in fluid communication therewith into the male urethra of the subject such that the treatment balloon is positioned in the lumen of the prostatic urethra, the prostatic urethra lumen having a wall and a cross-sectional width, and wherein the treatment catheter defines a portion of a closed loop thermal treatment system;
expanding the treatment balloon outwardly a distance to cause the treatment balloon to contact the wall of the prostatic urethra and exert pressure onto tissue proximate the prostatic urethra; and
substantially continuously circulating liquid heated to between about 45° to 95° C. through the liquid circulation path and the expanded treatment balloon for a time of at least about 5 minutes to heat tissue surrounding the prostatic urethra so to that a thermal therapy is administered thereto.

33. A method according to claim 30, further comprising:
monitoring the pressure in the closed loop system; and automatically adjusting the pressure in the closed loop system based on the pressure determined by the monitoring step.

34. A method according to claim 33, wherein the treatment catheter comprises an expandable treatment balloon, the method farther comprising automatically collapsing the treatment balloon before the catheter is removed from the subject at a desired point in the administration of the thermal therapy.

35. A method according to claim 33, wherein the step of adjusting the pressure is carried out by removing from or adding to the amount of liquid in the circulation path based on the monitoring step.

36. A method according to claim 32, further comprising directing body fluids to drain through the treatment catheter during the circulating step.

37. A method according to claim 27, further comprising generating an audible alarm when the cassette is improperly mounted to the console.

38. A method according to claim 27, wherein the console pump comprises a shaft that rotates, and wherein the cassette comprises a heating element that heats the liquid in the circulation flow path, the method further comprising monitoring at least one of the pump shaft rotation and the temperature of the heater to detect potential overheating conditions and generating an audible or visual alerting when such a condition is detected.

39. A method according to claim 29, wherein the cassette comprises a digital thermometer and wherein the disabling step is carried out by electrically disabling the digital thermometer.
